(19) United States
(12) Patent Application Publication
(10) Pub. No.: US 2010/0075435 A1
(43) Pub. Date: Mar. 25, 2010

(54) BIOTINYLATED THYROXINE

(76) Inventors: Noriyuki KASAGI, Kanagawa (JP); Tadahiro Matsuno, Kanagawa (JP)

Correspondence Address: BIRCH STEWART KOLASCH & BIRCH, PO BOX 747, FALLS CHURCH, VA, 22040-0747, US

(21) Appl. No.: 12/509,736

(22) Filed: Jul. 27, 2009

(30) Foreign Application Priority Data
Jul. 28, 2008 (JP) 2008-193072

(51) Int. Cl. G01N 33/53; C07D 495/04; C07K 14/77

(52) U.S. Cl. 436/500; 548/303.7; 530/367

(57) ABSTRACT

A means for conveniently and accurately measuring thyroxine or thyronine present in the blood or the like is provided: a compound represented by general formula (I) below:

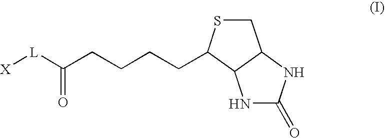

wherein L represents a linear or branched-chain linker having 5 to 50 total atoms; X represents one or more thyroxines, thyronines, or residues of derivatives thereof, bound to the main chain and/or side chain of the linker represented by L, a conjugate in the form of the above compound bonded to an avidin protein, and an aqueous dispersion including a microparticles the surface of which is modified with the above conjugate.

### CROSS-REFERENCE TO RELATED APPLICATIONS

This application claims benefit of priority under 35 USC 119 to Japanese Patent Application No. 2008-193072 filed on Jul. 28, 2008, the disclosure of which is expressly incorporated by reference herein in its entirety.

### TECHNICAL FIELD

The present invention relates to biotinylated thyroxine and thyronine.

### BACKGROUND ART

Thyroid hormones are hormones that are secreted by the thyroid gland and act on all cells in the body, such as by increasing the cellular metabolism rate. There are two known thyroid hormones: thyronine (triiodothyronine, sometimes abbreviated to “T3” in the present specification) and thyroxine (sometimes abbreviated to “T4” in the present specification). Most of the thyroid hormone circulating in the blood is T4. Recently, the number of patients with thyroid disorders, such as hyperthyroidism, where an excess of thyroid hormone is secreted (such as Basedow's disease), and hypothyroidism, where insufficient thyroid hormone is secreted (such as chronic thyroiditis (Hashimoto's disease)), has been increasing. There is a need to provide a means of conveniently and accurately measuring thyroid hormones during clinical examination.

Thyronine and thyroxine are characterized by extremely low solubility in water. Conjugates with water-soluble proteins (T4 or T3-protein conjugates) are employed in bioassays. To this end, for example, the method of N-hydroxysuccinimide esterifying a terminal carboxyl group to modify an amino group of a protein has been adopted (U.S. Pat. No. 6,527,709, the disclosure of which is expressly incorporated by reference herein in its entirety). However, when this method is applied to a microparticulate dispersion (such as polystyrene beads the surfaces of which have been modified with carboxyl groups) using charge repulsion instead of a protein, aggregation is caused by the elimination of the charge on the surface of the microparticulate dispersion, creating a problem in that modification with T4 or T3 cannot be conducted while ensuring adequate dispersibility of the particles.

The method of immobilizing avidin or streptavidin to the surface of the microparticles and exploiting the reaction with biotin to secure a targeted substance is generally employed. However, Nature Structural Biology, 9, 582 (2002) and the J. Am. Chem. Soc., 129, 873 (2007), the disclosures of which are expressly incorporated by reference herein in their entireties, state that the biotin-binding site present on streptavidin is positioned far from the surface (at about 27 Angstroms) and a suitable spacer needs to be inserted.

### SUMMARY OF THE INVENTION

The object of the present invention is to provide a means for conveniently and accurately measuring thyroxine or thyronine present in the blood or the like. More specifically, the object of the present invention is to provide a means for modifying the surface of microparticles with thyroxine or thyronine without losing the water dispersibility of the microparticles, such as polystyrene beads.

The present inventors conducted extensive research into achieving the above objects. As a result, they found that disposing an avidin protein such as avidin or streptavidin on the surface of microparticles such as polystyrene to form a hydration layer, and binding the specific biotinylated thyroxine or thyronine indicated below to the avidin protein permitted the modification of the surface of the microparticles with thyroxine or thyronine without loss of the dispersibility of the microparticles in water. The present invention was devised based on this finding.

The present invention thus provides a compound represented by general formula (I) below:

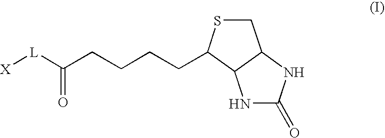

(wherein L represents a linear or branched-chain linker having 5 to 50 total atoms; X represents one or more thyroxines, thyronines, or residues of derivatives thereof, bound to the main chain and/or side chain of the linker represented by L).

According to preferred embodiments, the present invention provides the above compound in which the linker represented by L includes at least one chain-like structure selected from among the group consisting of an alkyl chain, a polyethylene glycol chain, and a peptide chain; the above compound in which L includes one or more bonds selected from the group consisting of an amide bond, an ester bond, and a carbamate bond in addition to an alkyl chain and/or a polyethylene glycol chain; the above compound in which the main chain of the linker represented by L consists of 15 to 20 atoms; the above compound in which the main chain of the linker represented by L is 25 to 30 Angstroms in total length; the above compound wherein the residue is a residue obtained by removing the hydrogen atom from a carboxyl group of thyroxine, thyronine, or a derivative thereof, or a residue obtained by removing one of the hydrogen atoms in the amino group of thyroxine or thyronine; the above compound wherein the derivative is a carboxylic acid ester derivative or an amino group-modified derivative; and the above compound having an interaction with an avidin protein represented by a Kd value of 1×10⁻¹³ or lower.

From another aspect, the present invention provides a conjugate in the form of the compound represented by general formula (I) above bonded to an avidin protein. According to preferred embodiment, the present invention provides the above conjugate wherein the avidin protein is avidin, streptavidin, or neutra-avidin.

The present invention further provides an aqueous dispersion, including a microparticle the surface of which is hydrophilically treated, wherein the surfaces of the microparticle is modified with a conjugate comprising the compound represented by general formula (I) above bound to an avidin protein. According to preferred embodiment, the present invention provides the above aqueous dispersion wherein the microparticle is a carboxy hydrophilically-treated polystyrene bead, and the above aqueous dispersion wherein the avidin protein moiety of the conjugate is contained in a hydration layer formed on the surface of the carboxy hydrophilically-treated polystyrene bead. The present invention further provides the above aqueous dispersion employed for a measurement of thyroxine or thyronine.

From still another aspect, the present invention provides a method for measuring thyroxine or thyronine using the above conjugate or aqueous dispersion; a method for measuring antibodies to thyroxine or thyronine using the above conjugate or aqueous dispersion; and a method for purifying thyroxine or thyronine, or antibodies to thyroxine or thyronine, using the above conjugate or aqueous dispersion.

### BRIEF DESCRIPTION OF THE DRAWINGS

FIG. 1 is a plot of the results of measurement of T4 under conditions D with the compound obtained in Example 1.

FIG. 2 is a plot of the results of calculating the optimal labeling rate in a competitive assay employing the compound obtained in Example 1.

FIG. 3 shows plots of the results of SPR measurement when adding antibodies 1 to 4 to sensor chips on which the compound obtained in Example 1 had been immobilized.

### BEST MODES OF CARRYING OUT THE INVENTION

In general formula (I) above, X represents a residue of thyroxine, thyronine, or a derivative thereof. In the present specification, the term “residue” means the partial structure remaining after removing a hydrogen atom from thyroxine or thyronine; preferably means the partial structure remaining after removing a hydrogen atom from the amino group or a carboxyl group of thyroxine or thyronine; and more preferably means the partial structure remaining after removing one of the two hydrogen atoms of the amino group of thyroxine or thyronine.

In the present specification, the derivatives of thyroxine or thyronine are not specifically limited. Preferable examples include derivatives obtained by modifying the carboxyl group or the amino group of thyroxine or thyronine. For example, compounds obtained by esterifying a carboxyl group of thyroxine or thyronine are preferred. Alkyl ester derivatives can be suitably employed, for example. Examples of alkyl ester derivatives include methyl ester derivatives and ethyl ester derivatives. Derivatives obtained by alkylating or acylating the amino group of thyroxine or thyronine can also be preferably employed. For example, derivatives obtained by methylating or ethylating a carboxyl group, and derivatives obtained by acetylating or benzoylating the amino group are preferred.

L represents a linear or branched-chain linker comprising a total of 5 to 50 carbon atoms. Part or all of the linker represented by L may include at least one chain structure selected from the group consisting of an alkyl chain, a polyethylene glycol chain, and a peptide chain. A peptide chain in the form of a dipeptide chain, tripeptide chain, tetrapeptide chain, or an oligopeptide chain consisting of about 5 to 10 amino acid residues may be employed. One or more bonds selected from the group consisting of an amide bond, an ester bond, and a carbamate bond may be contained, and one amino acid may be contained as a linker structural unit.

The entire linker is preferably consisting of an alkyl chain or polyethylene glycol chain. From the perspective of imparting a hydrophilic property, the linker particularly preferably consists of a polyethylene glycol chain. A chainlike linker may include one or more branch chains. When two or more branch chains are present, they may be identical or different. For example, a methyl group and an ethyl group may be present as branch chains. The atoms constituting the linker are not specifically limited, and are preferably atoms selected from the group consisting of carbon atoms, oxygen atoms, nitrogen atoms, sulfur atoms, and hydrogen atoms, for example. A cyclic structure may be contained as a partial structure of the linker. Such a cyclic structure may be a nonaromatic cyclic structure (such as a cyclohexanediyl group, cyclohexenediyl group, or cyclene structure) or an aromatic cyclic structure (such as a phenylene or pyridinediyl).

The number of atoms contained in the main chain of the linker is not specifically limited, and may be about 15 to 20 atoms, for example. In the present specification, the term “main chain” of the linker means the shortest chain structure running from the atom at one end of the linker (bonded to the carbonyl group) to the atom at the other end (bonded to X). The number of atoms contained in the main chain means the smallest number of atoms from the atom at one end of the linker to the atom at the other end in the chain structure constituting the main chain. For example, the main chain of the linker represented by —CH₂—CH₂—O—CH(CH₂CH₃)—CH₂—O— is the chain structure —CH₂—CH₂—O—CH—CH₂—O—. Six atoms are contained in the main chain. The main chain of the linker represented by —CH₂—CH₂—O—CH(CH₂CH₃)—CH₂—C₆H₄—O— (where C₆H₄ represents a p-phenylene group) is —CH₂—CH—O—CH—CH₂—C₆H₄—. Ten atoms are contained in the main chain (there are four atoms from one end of the bond portion of the p-phenylene group to the other end). When a branch chain is present, in addition to the X bonded to the main chain, an X of the branch chain may also be bonded. When plural branch chains are present, an X can be bonded to one or more of them, or two or more Xs can be bonded to a single branch chain. The branch chains may have further branches.

The total length of the linker represented by L is not specifically limited. However, by way of example, it is preferably about 25 to 30 Angstroms, more preferably about 27 Angstroms. The total length of the main chain of the linker can be conveniently estimated by a method such as assembling a molecular model. Although not based on any specific theory, the groove of the biotin bond site in the avidin protein is known to have a depth of about 27 Angstroms (J. Am. Chem. Soc., 129, 873, 2007), and it is preferable for the total length of the main chain of the linker represented by L to be roughly identical to the depth of this groove. Further, the compound of the present invention preferably has a level of interaction Kd with an avidin protein of 1×10⁻¹³ M or lower. In the present specification, the term “avidin protein” represents a concept that includes avidin, streptavidin, and neutral avidin.

Preferable examples of the compound of the present invention are given below. However, the present invention is not limited to these compounds. In the formulas, Me represents a methyl group, Et represents an ethyl group, Pr represents an n-propyl group, i-Pr represents an isopropyl group, t-Bu represents a tert-butyl group, Ac represents an acetyl group, and Boc represents a tert-butoxycarbonyl group.

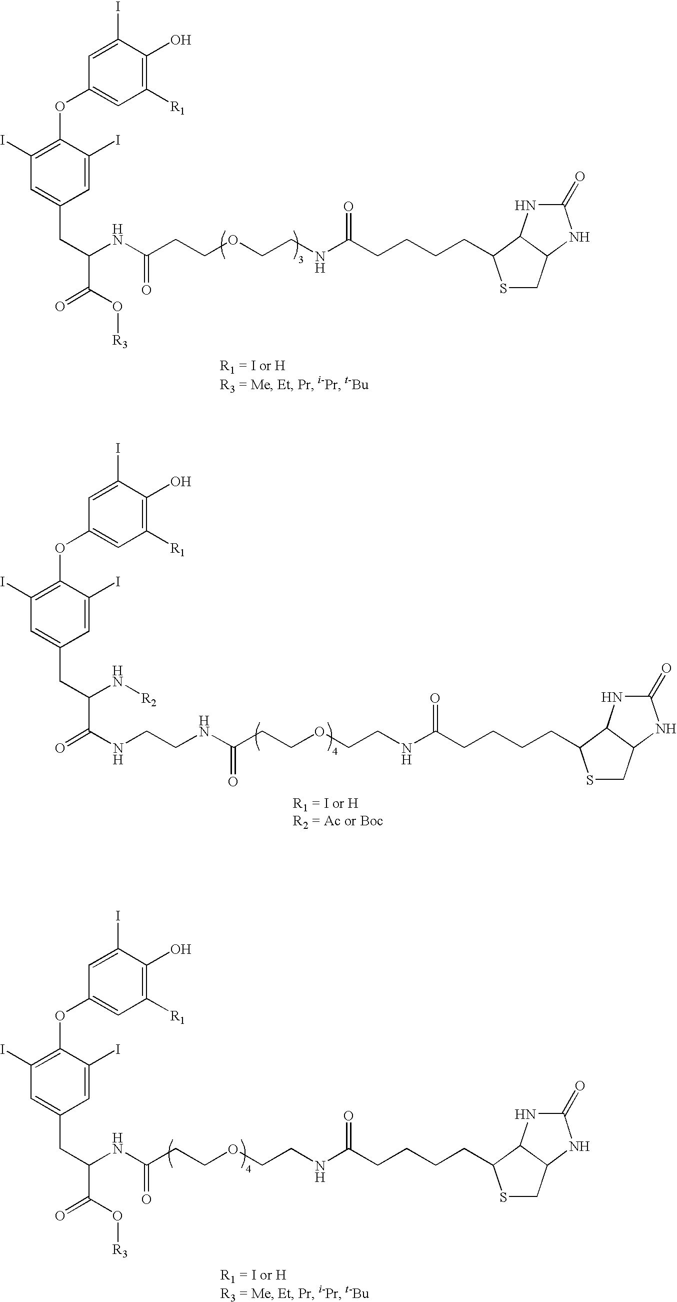

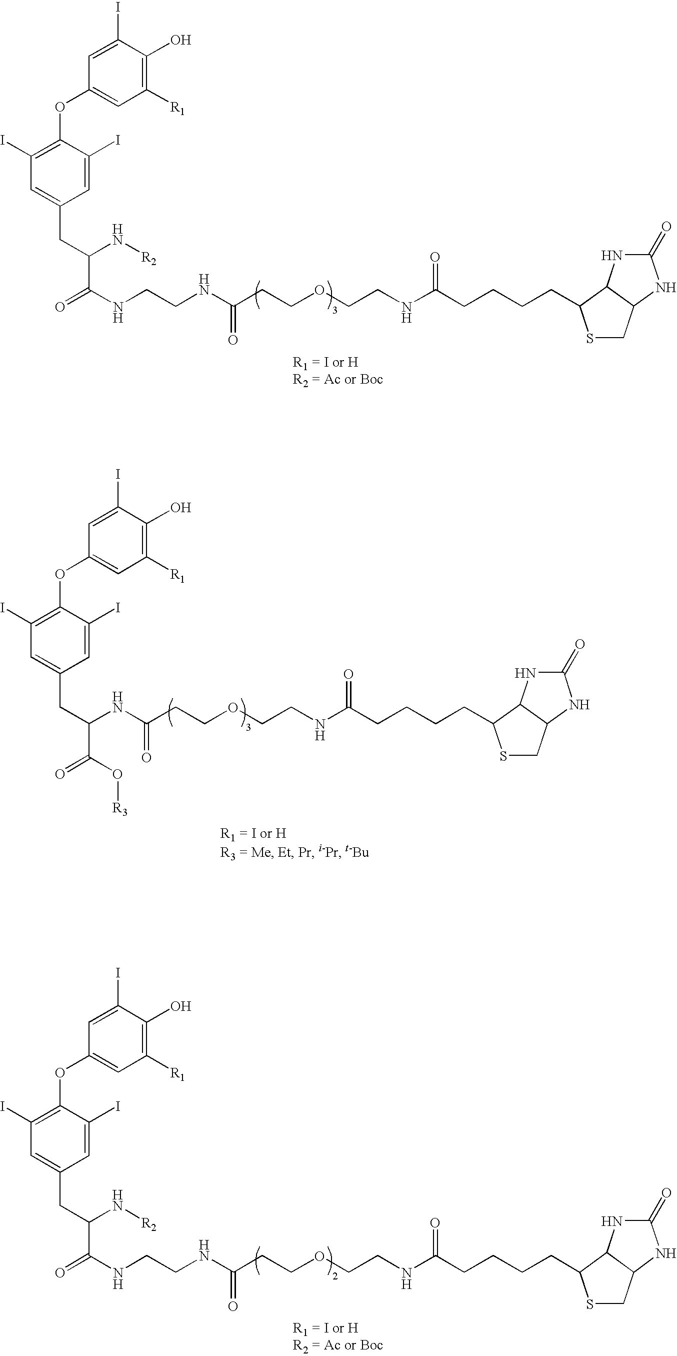

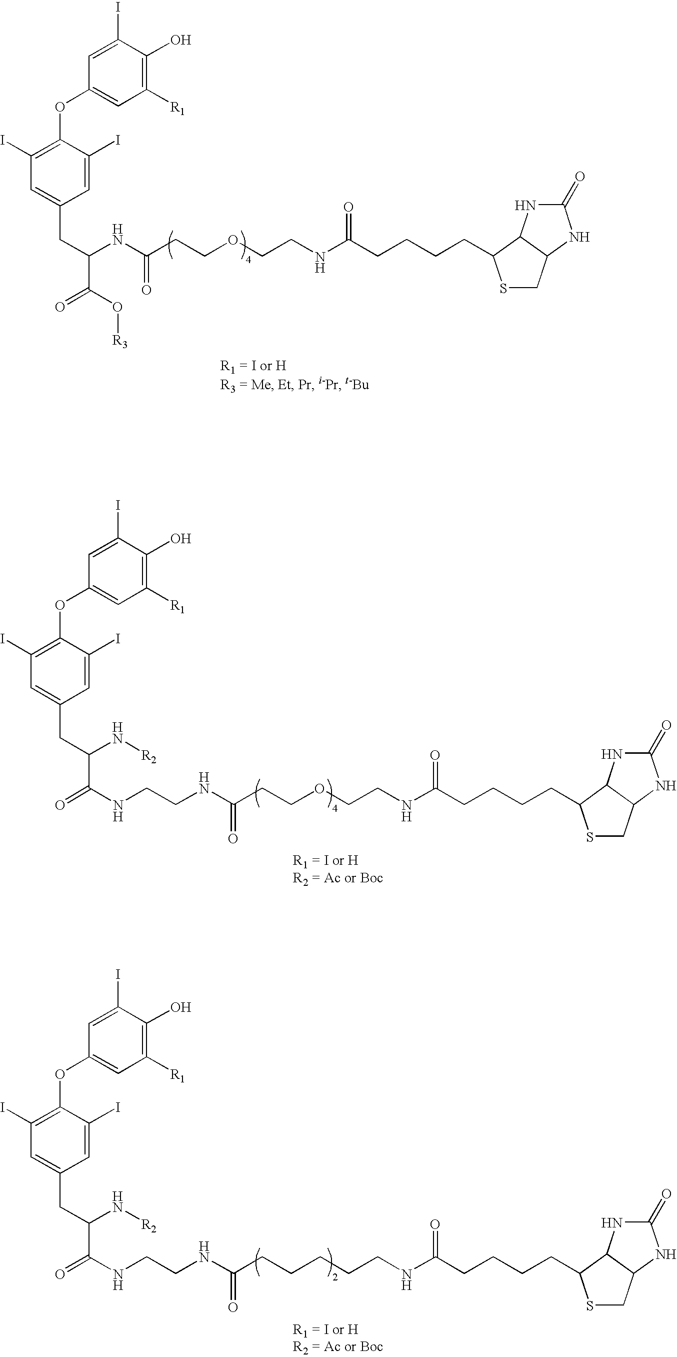

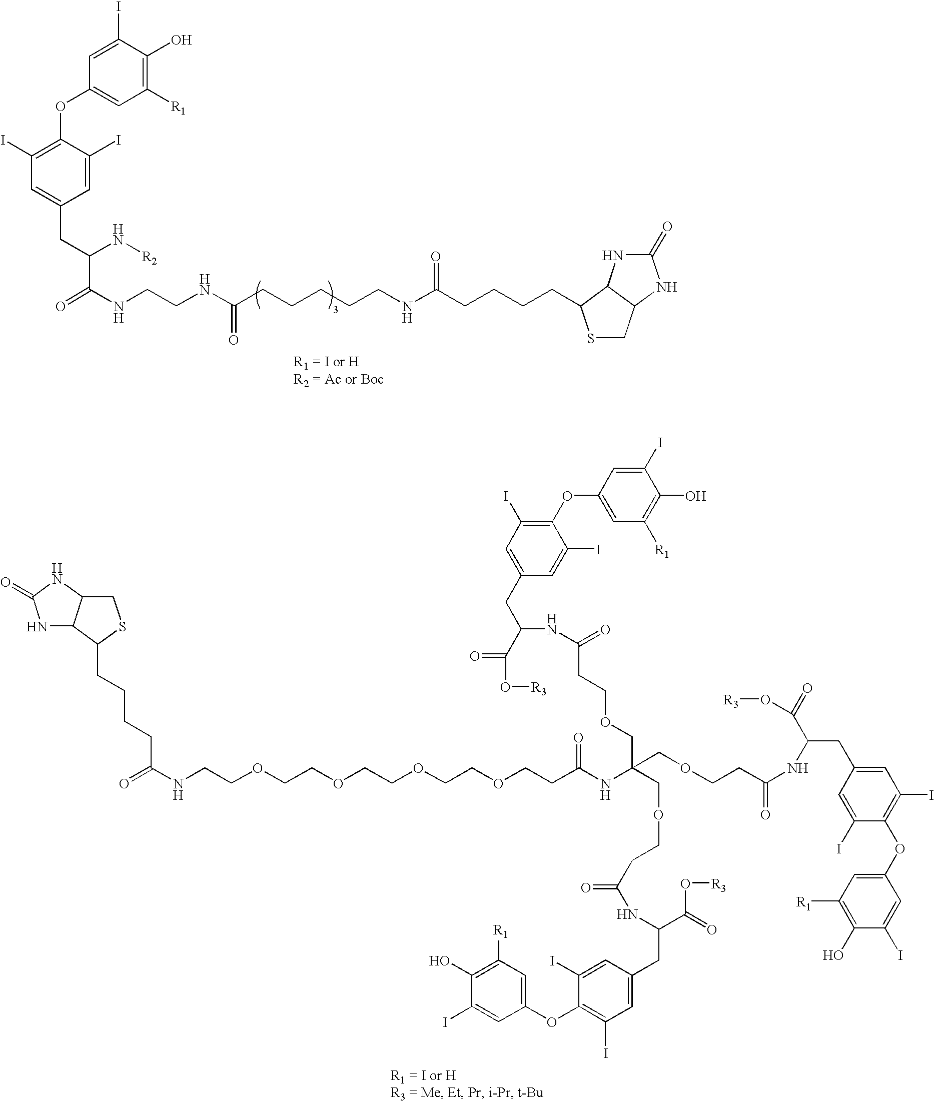

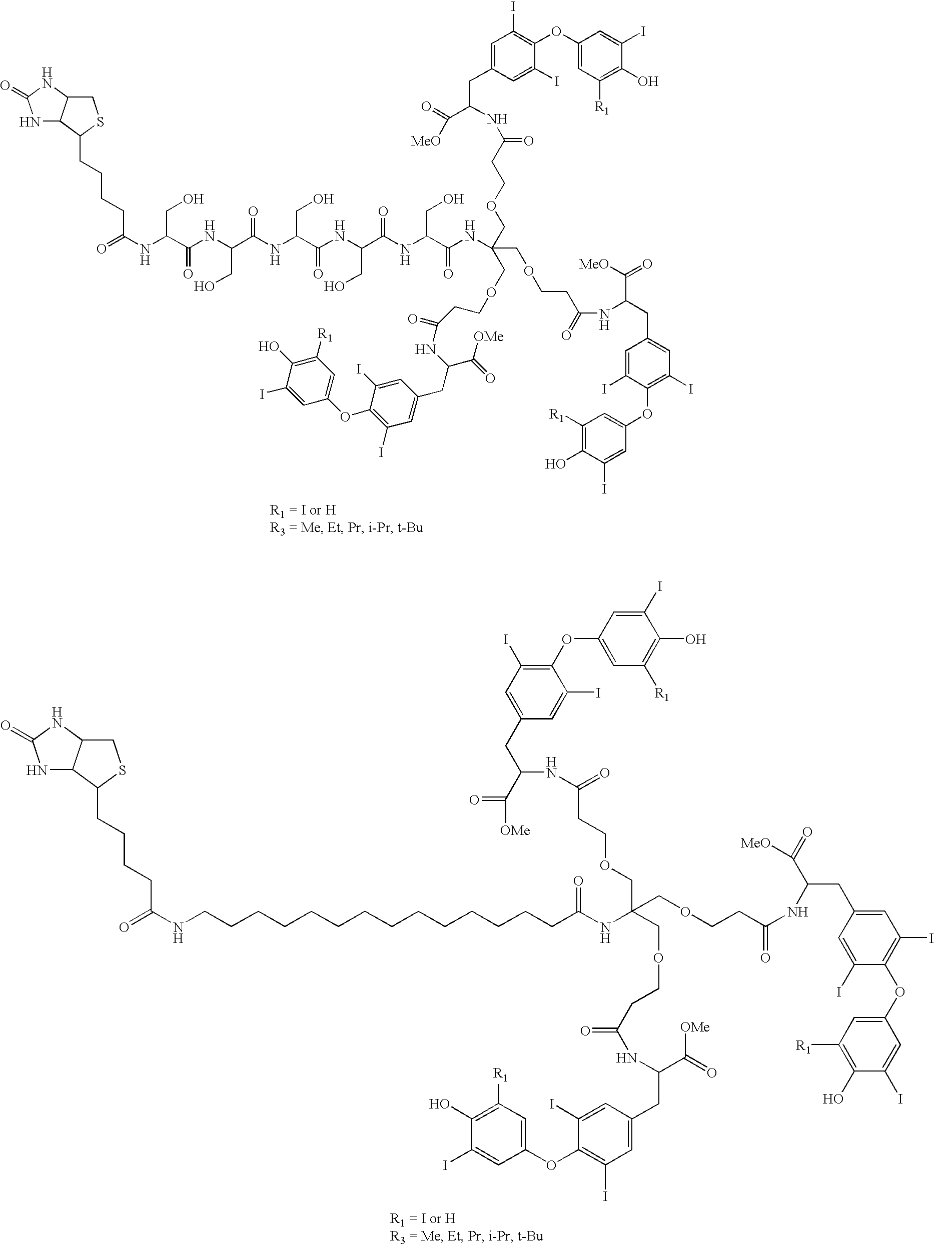

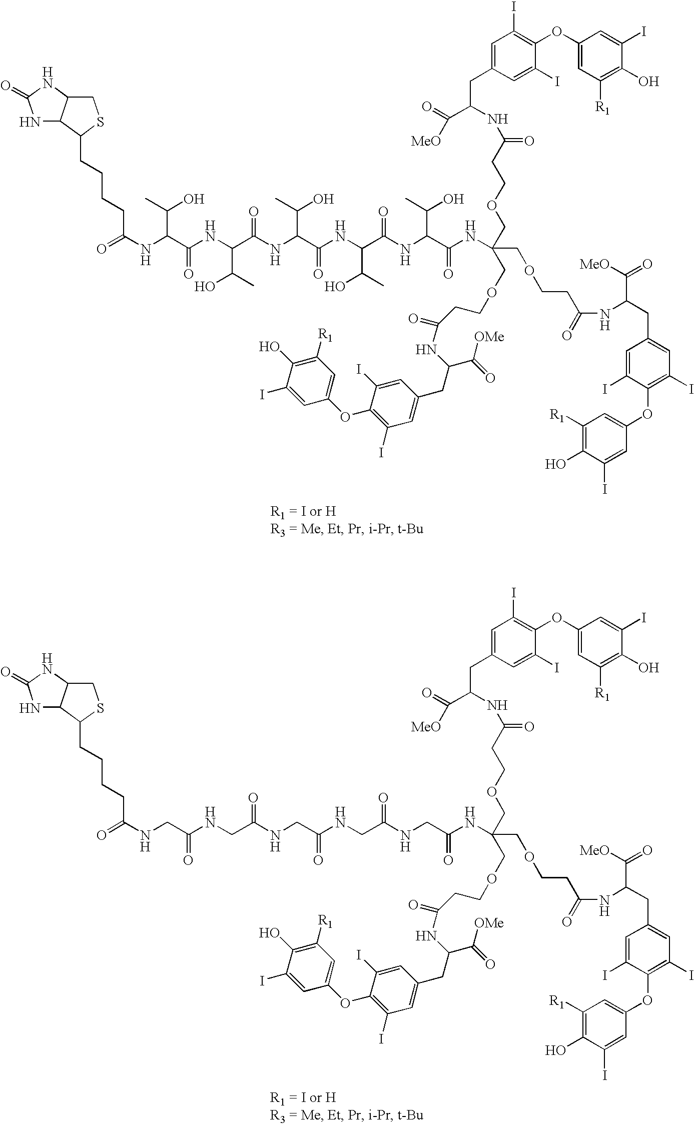

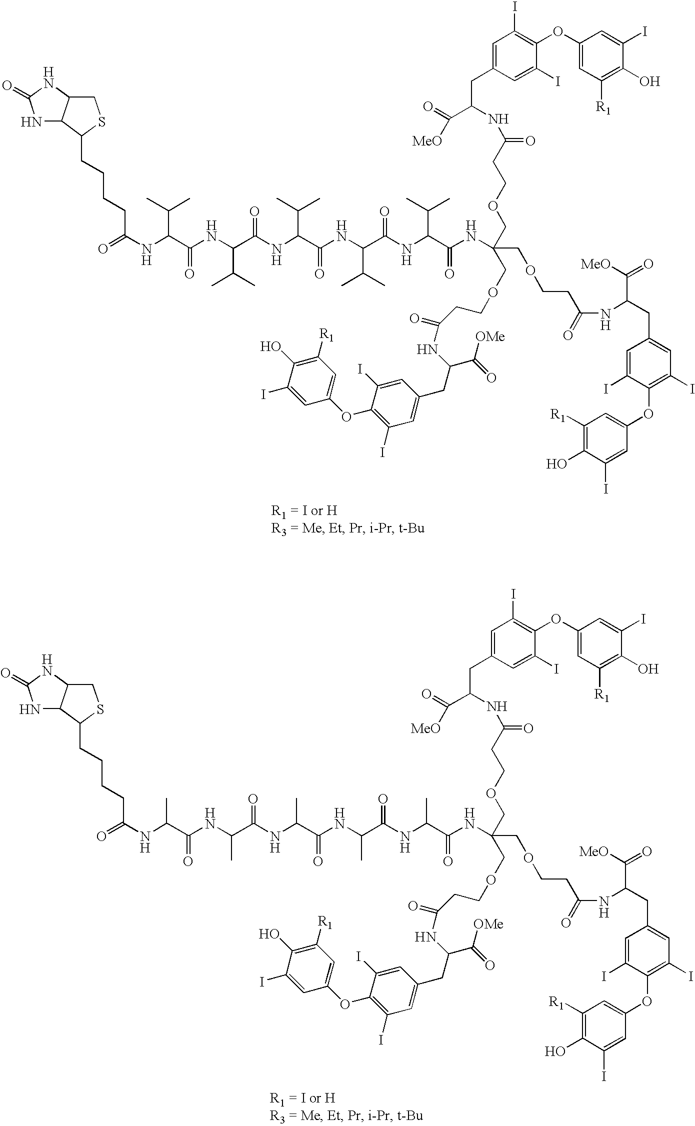

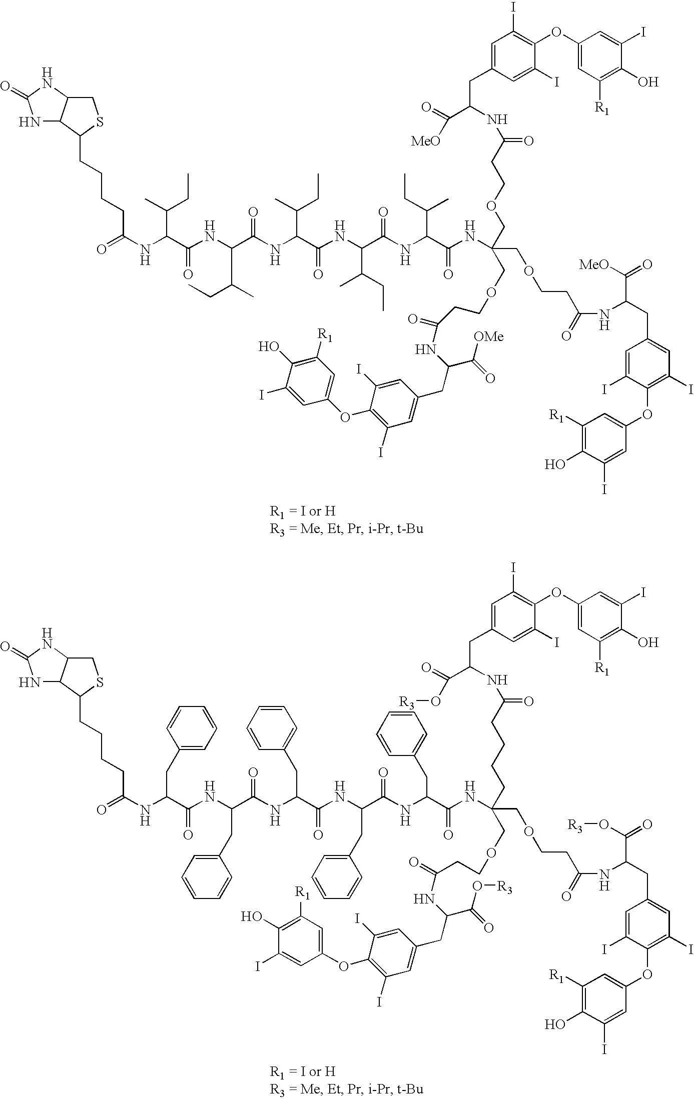

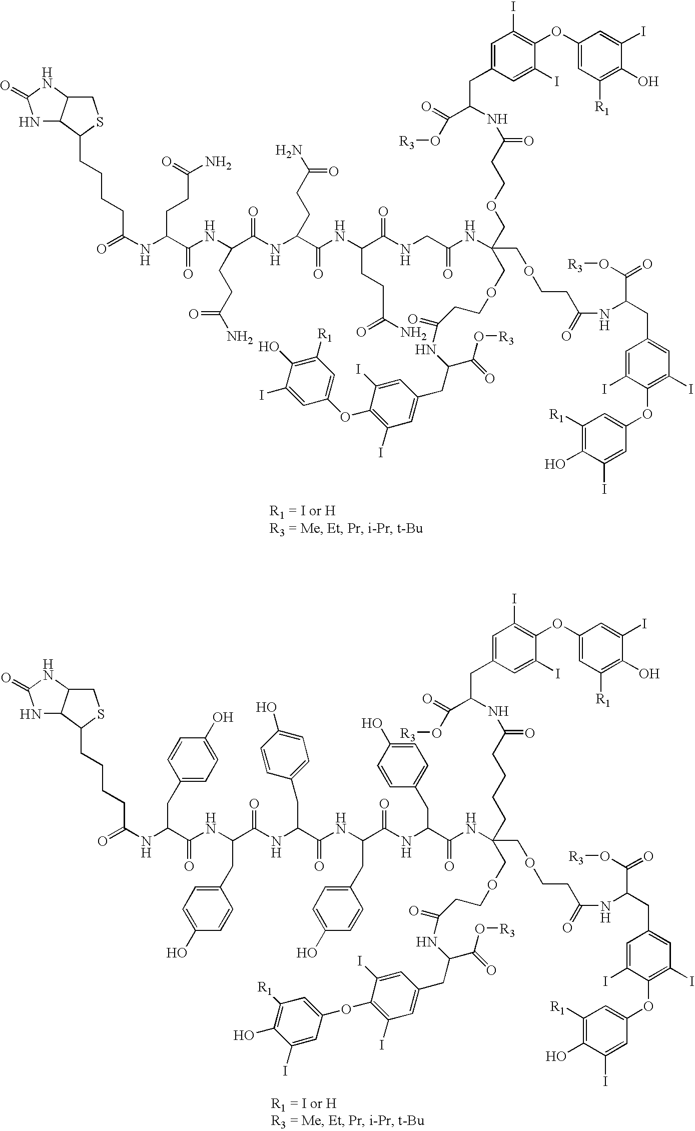

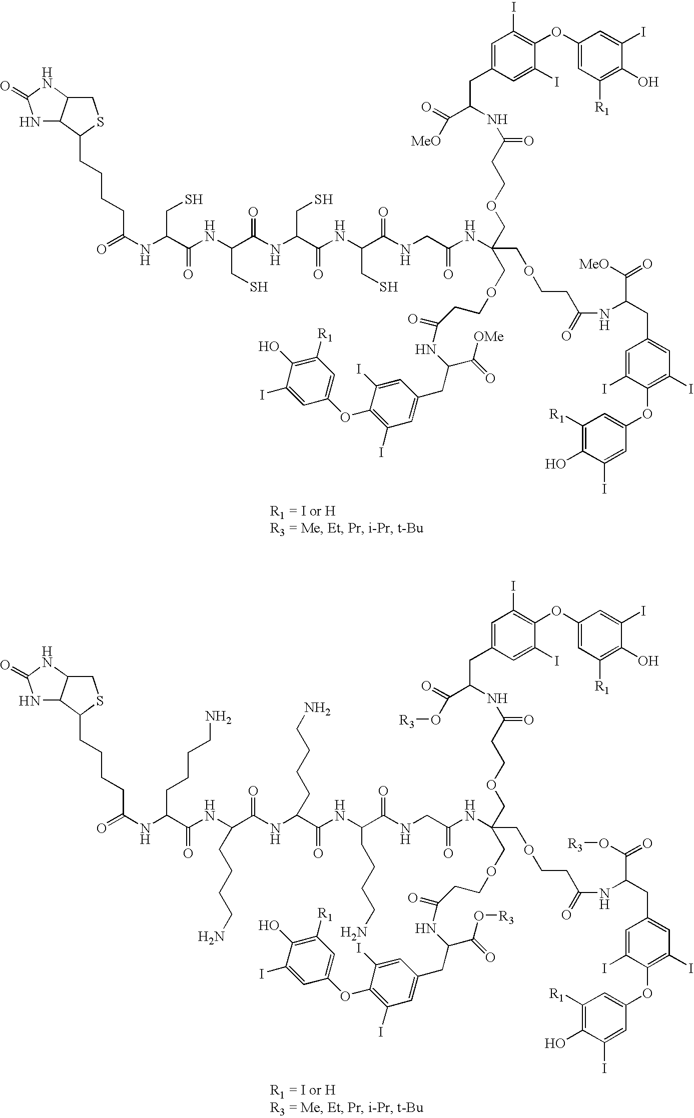

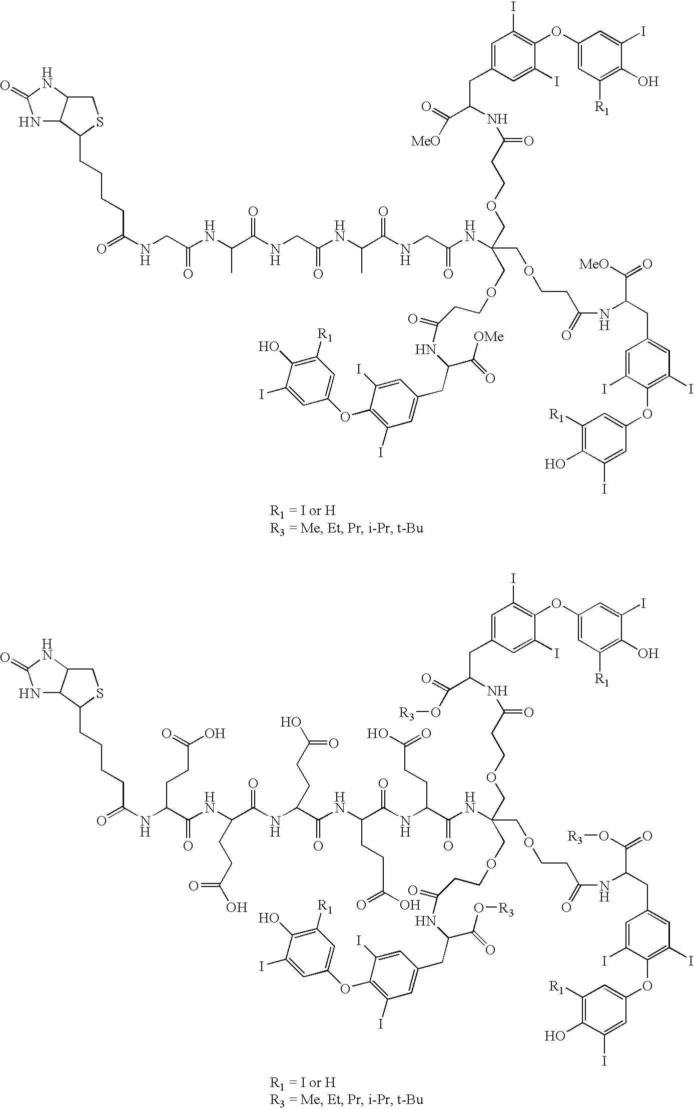

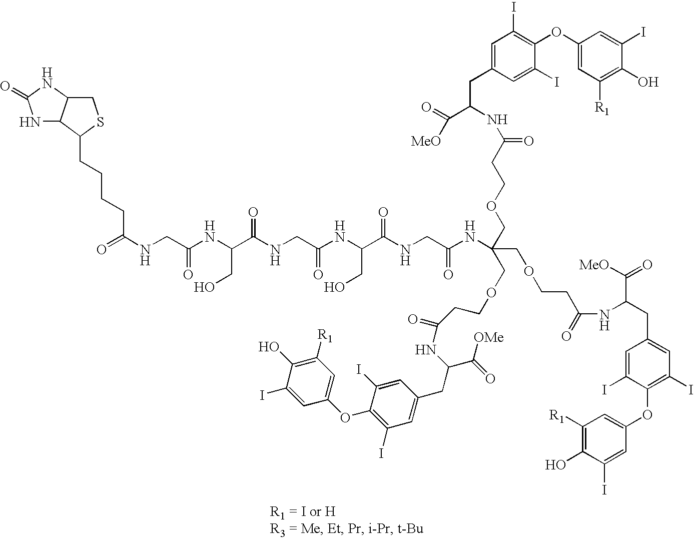

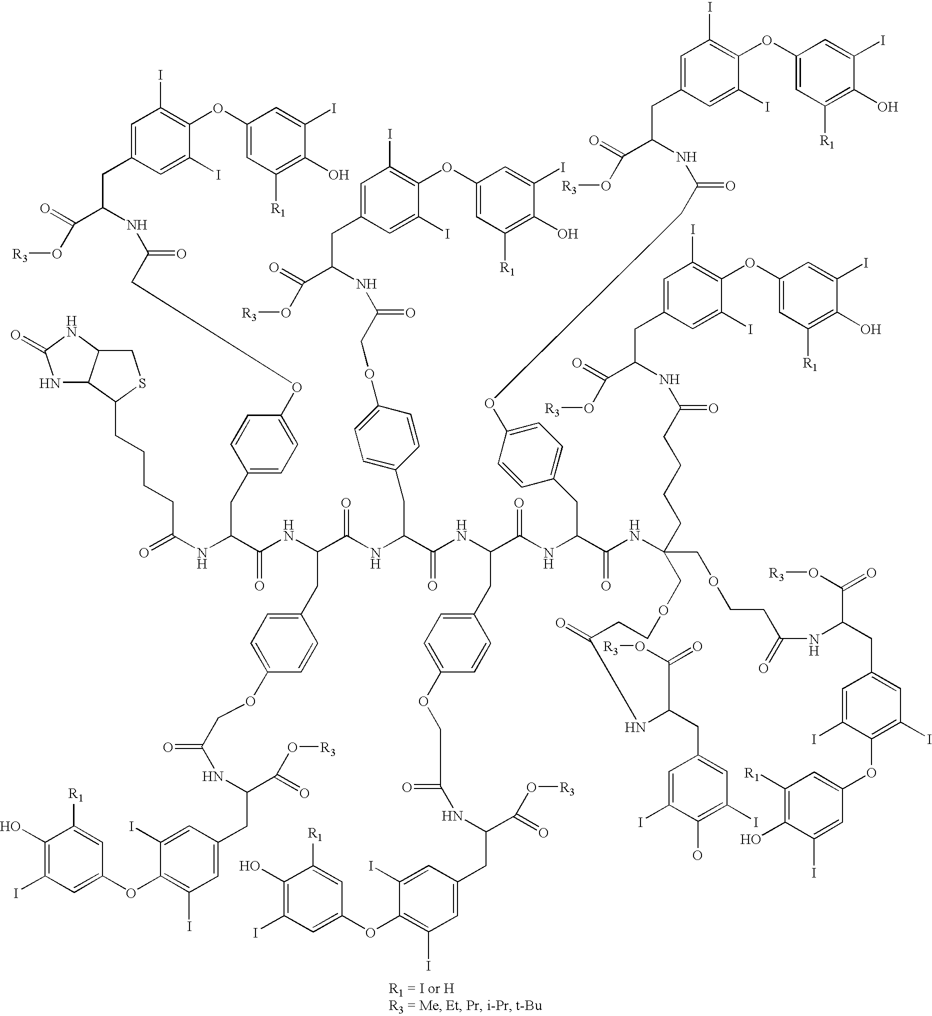

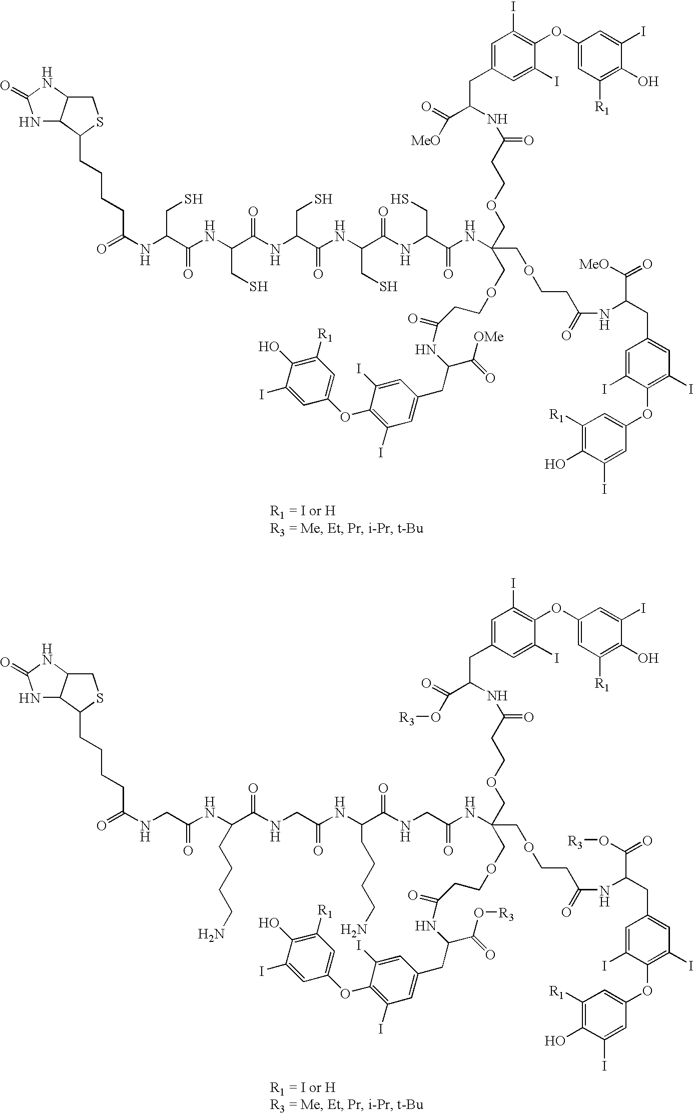

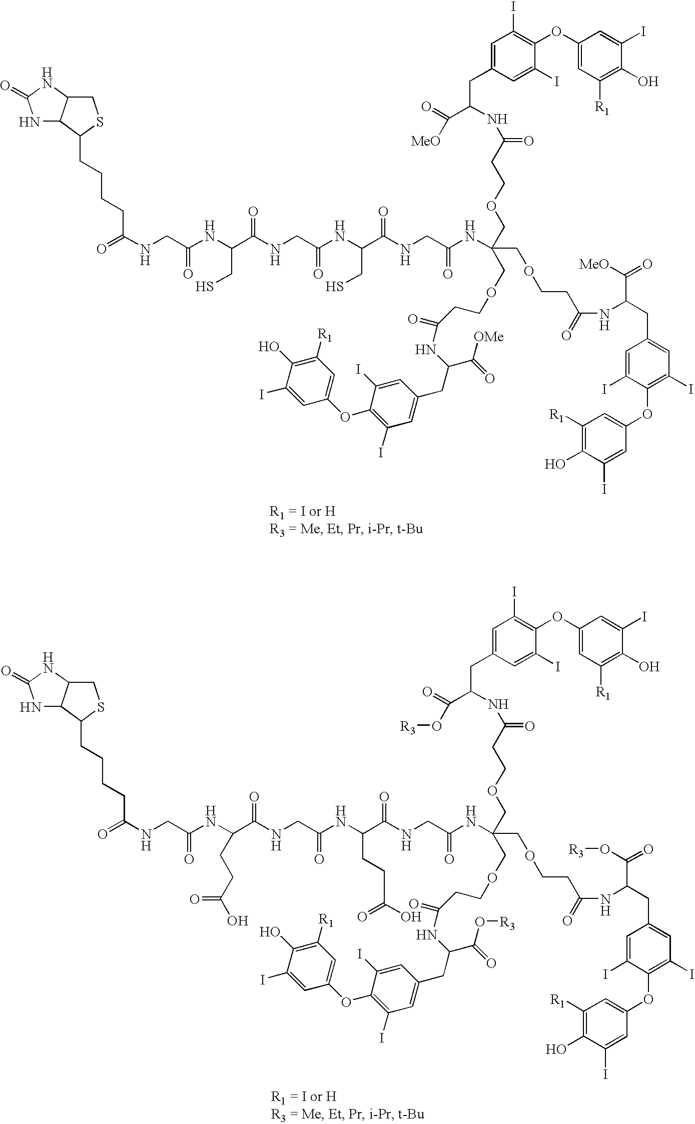

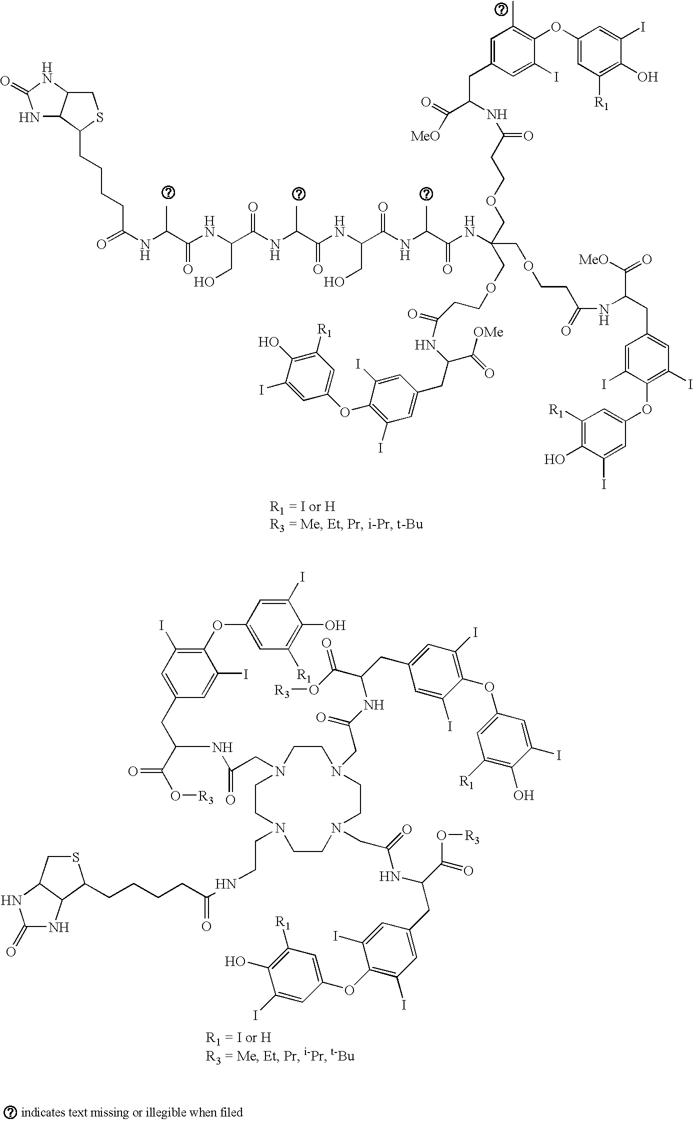

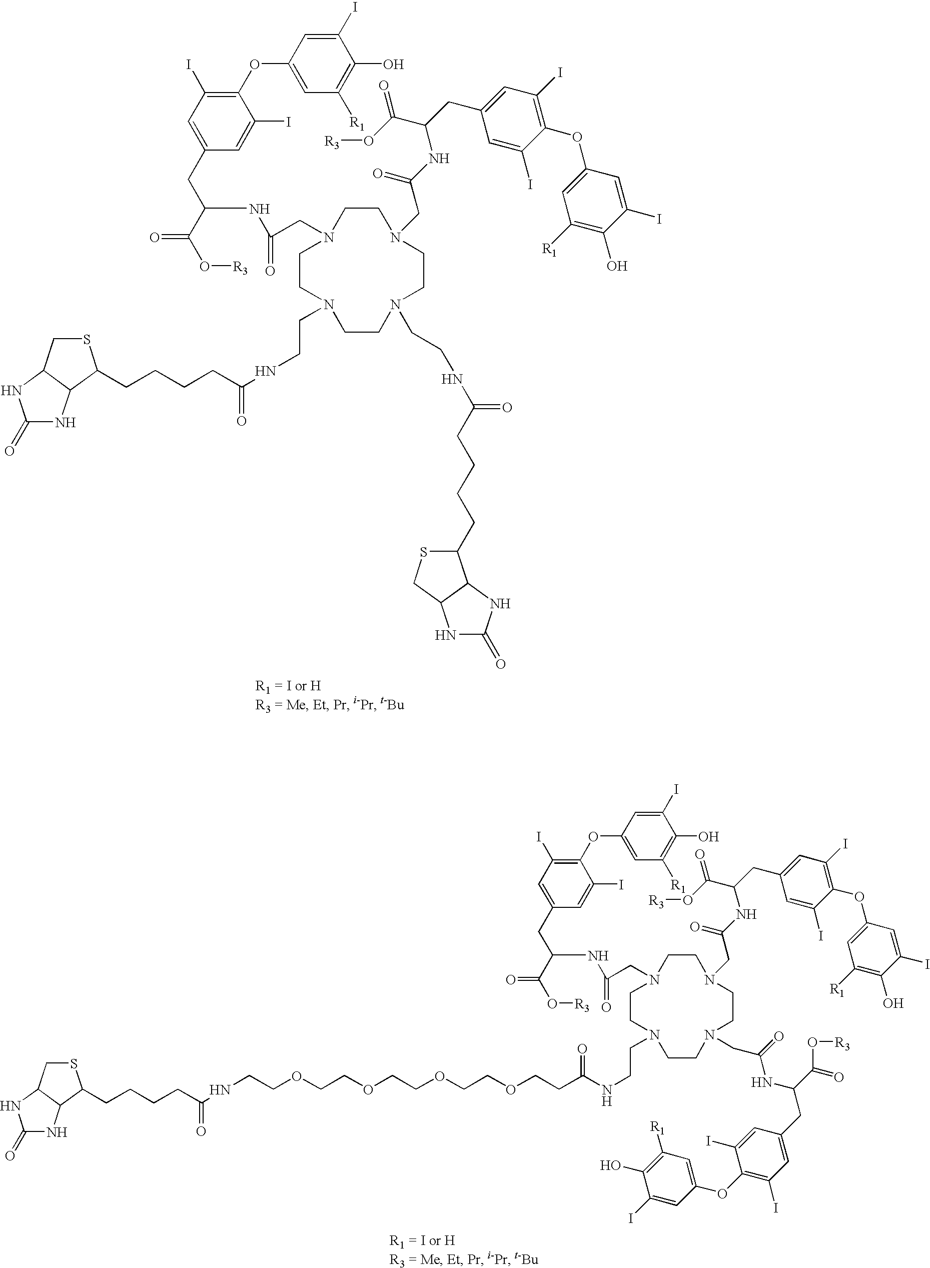

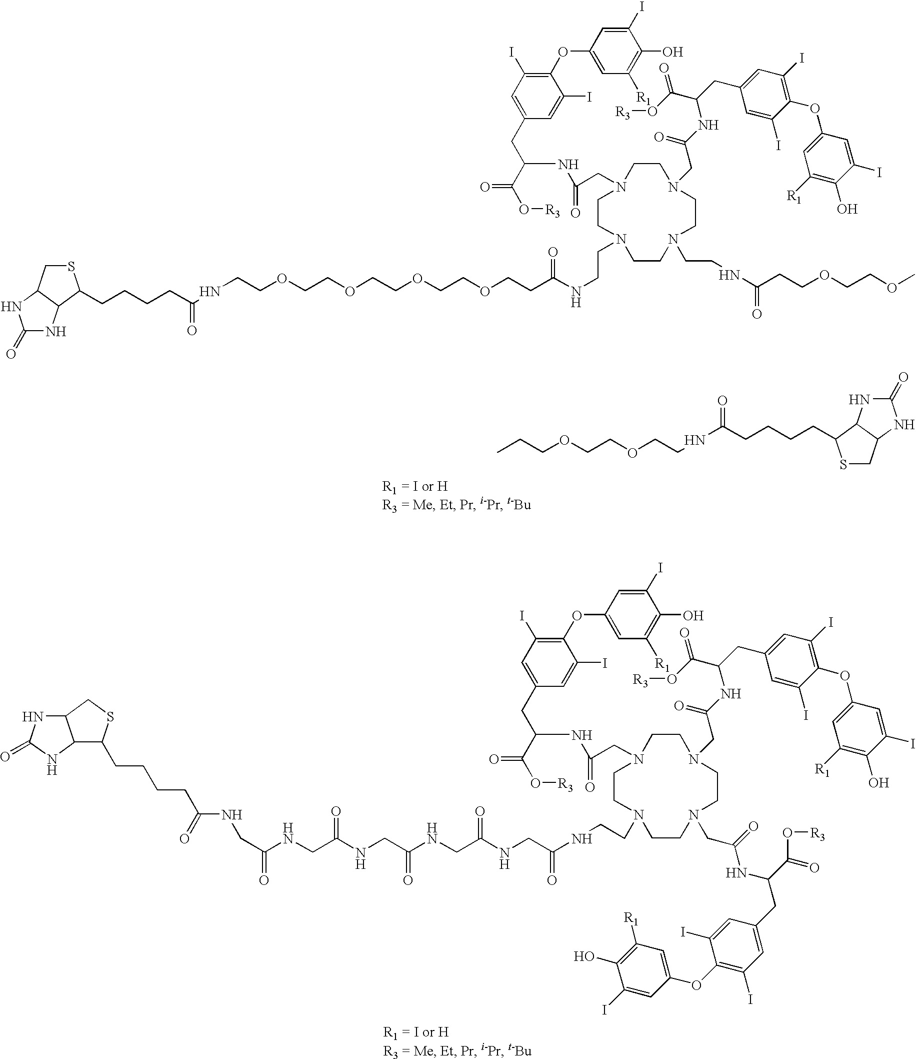

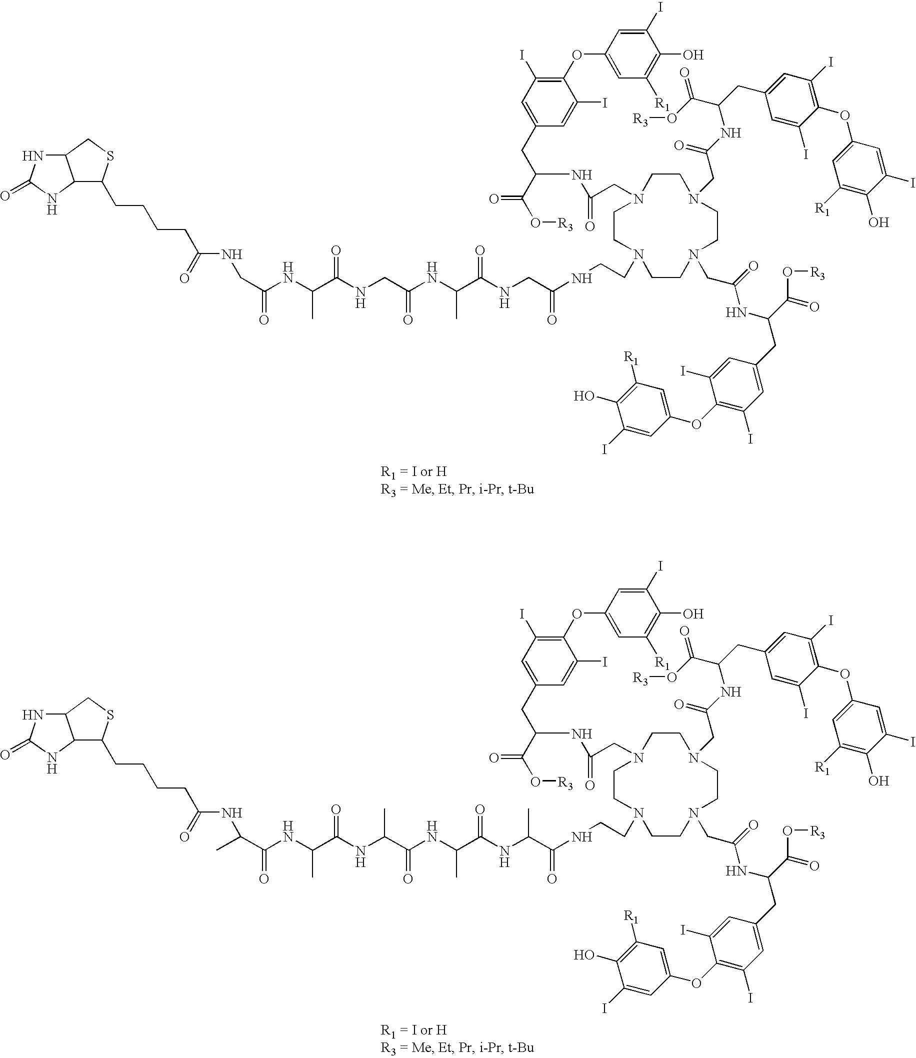

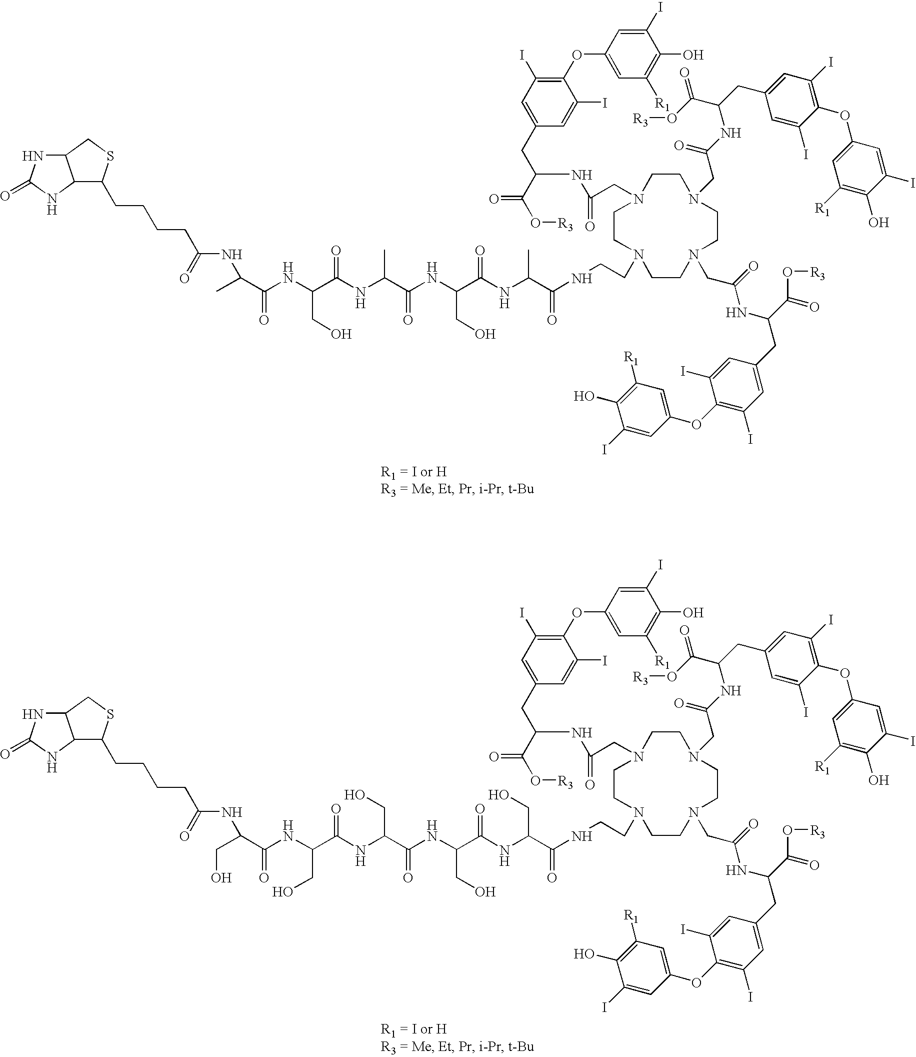

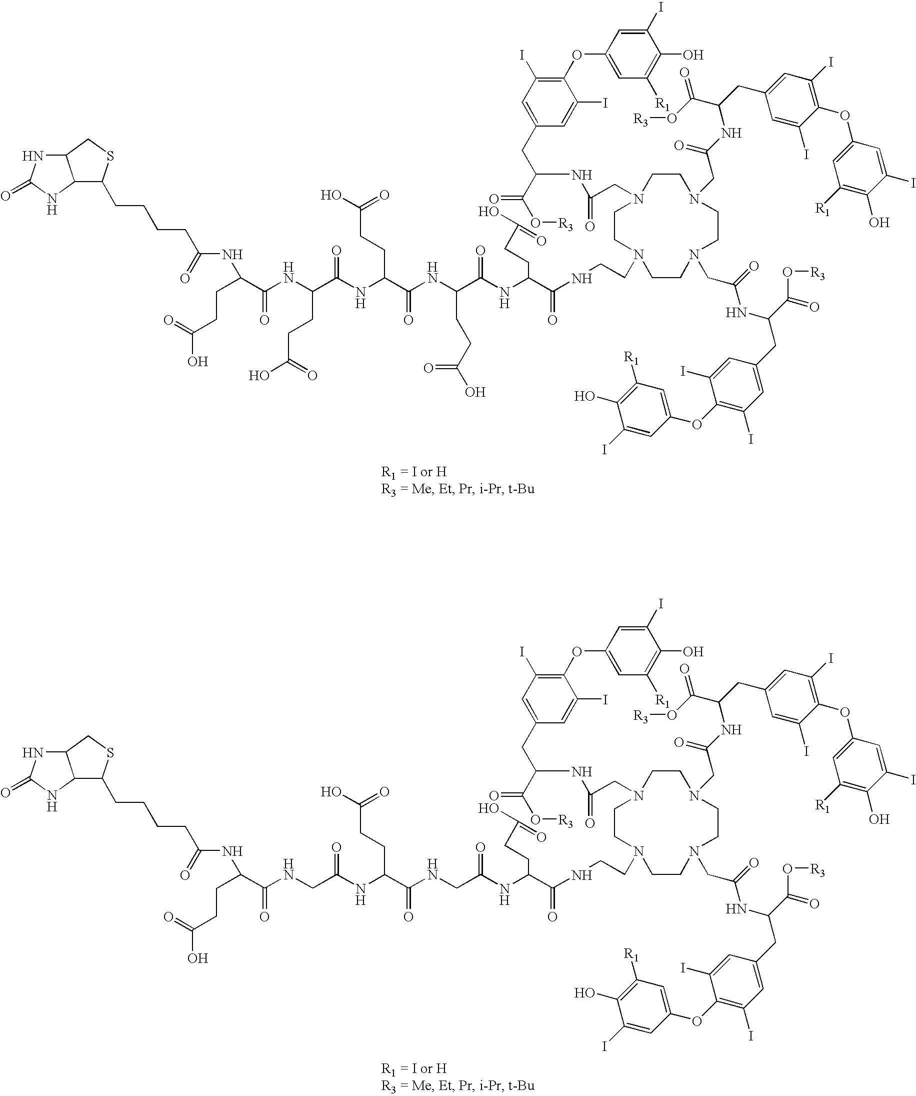

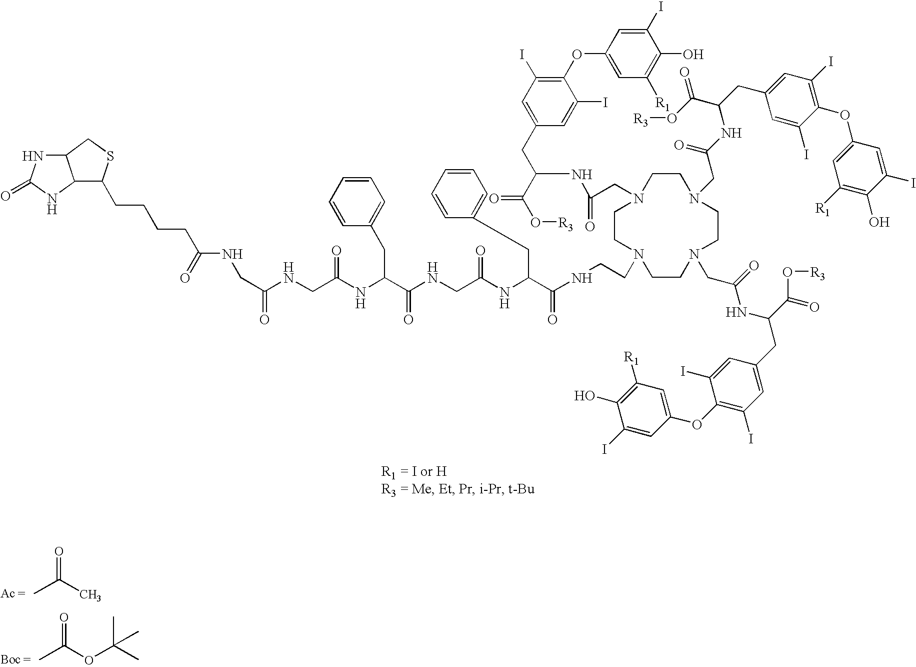

The method of manufacturing the compound of the present invention is not specifically limited. However, the compound of the present invention can generally be readily synthesized by the manufacturing methods set forth in the schemes below. Methyl esters of T4 and T3 can be manufactured by the synthesis method described in Bull. Chem. Soc., 52, 1879 (1979), the disclosure of which is expressly incorporated by reference herein in its entirety, or by suitably modified methods based on this method. The compound of the present invention can be manufactured by t-Bu ester deprotection under acidic conditions and condensation with the methyl ester of T4 or T3.

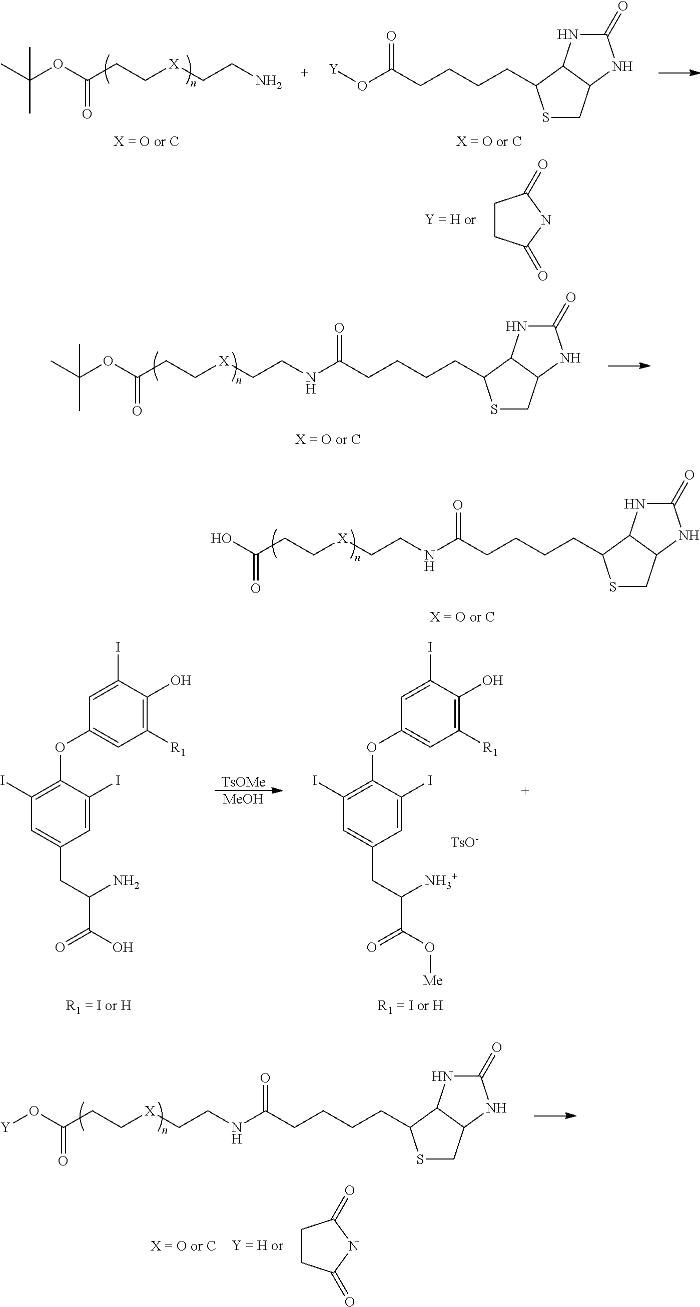

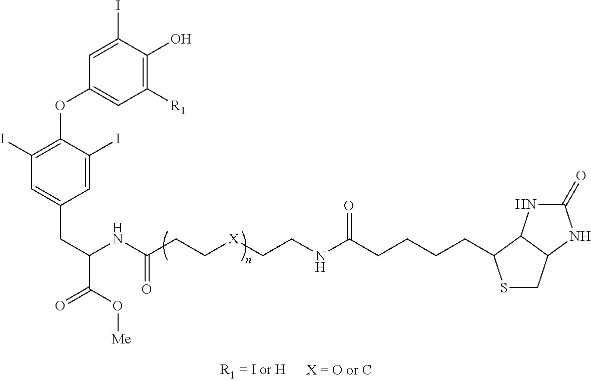

Further, the compound of the present invention can be manufactured by reacting T4 or T3 with acetic anhydride or Boc₂O to protect the amine, condensing the product with ethylene diamine, and condensing this product with a biotin derivative in the same manner as in the above-described manufacturing method. Based on these general manufacturing methods and the specific manufacturing methods described in the embodiments, as needed, the starting material compounds, reagents, reaction conditions, and the like can be suitably altered or modified by a person having ordinary skill in the art to readily manufacture the compound of the present invention. The method of manufacturing the compound of the present invention is not limited to these methods.

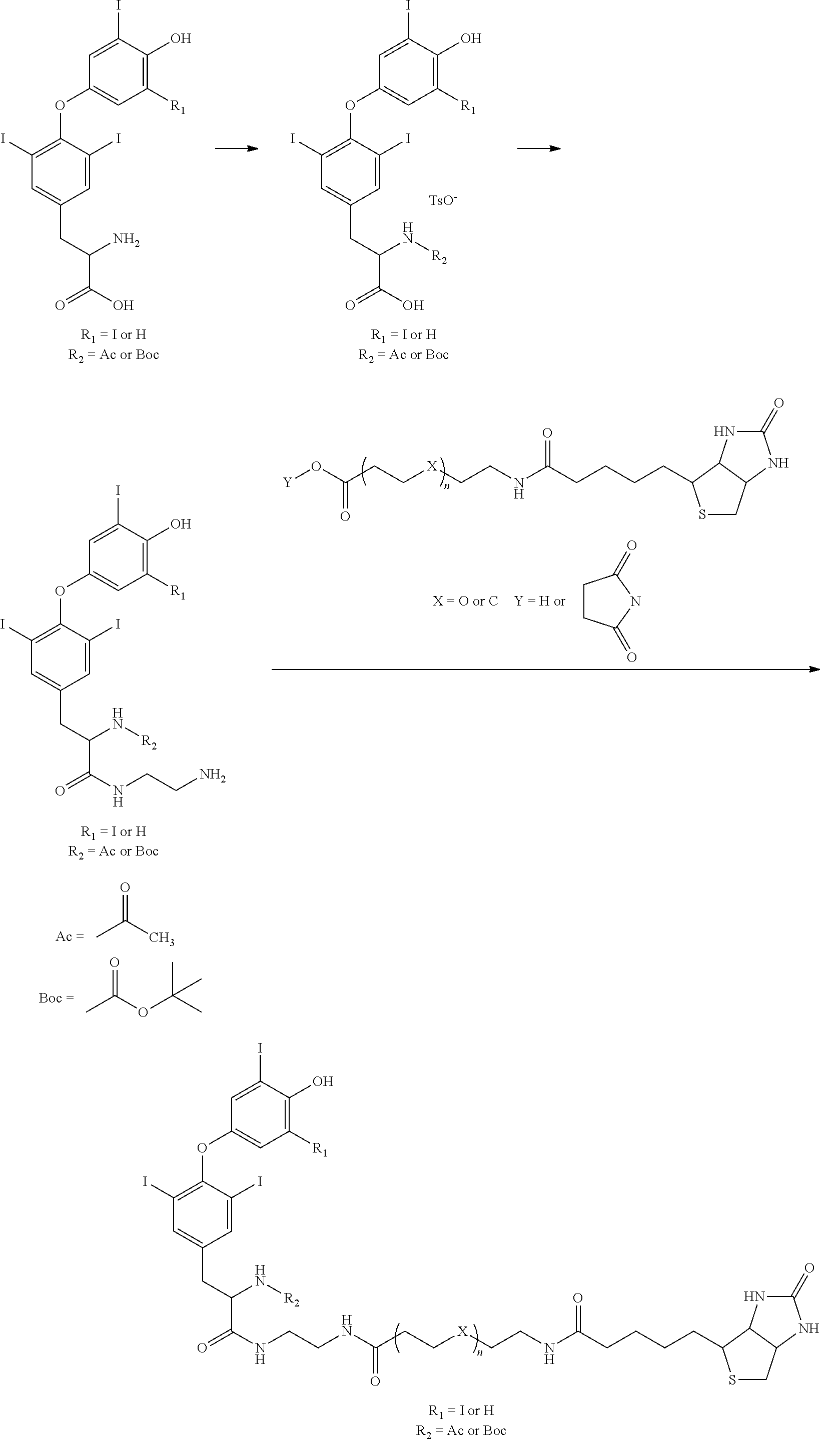

The compound represented by the following formula is an example of a preferred compound. However, the compound of the present invention is not limited to the compound given below. (In the formula, R¹ represents a hydrogen or iodine atom, preferably an iodine atom.)

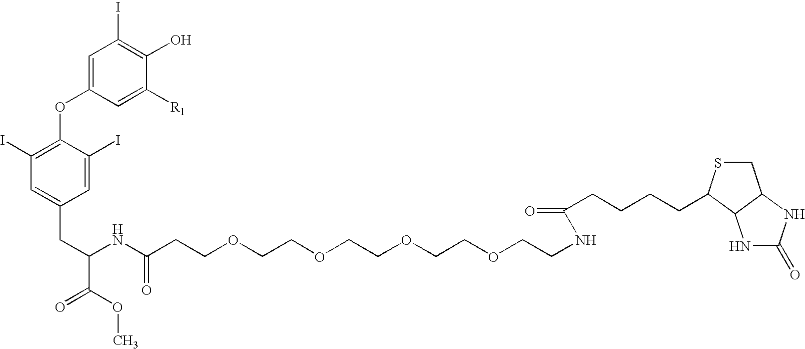

When the compound represented by general formula (I) above is reacted with an avidin protein, the biotin residue present in the compound of the present invention binds to the biotin-binding site present on the avidin protein, producing the conjugate of the present invention. By way of example, avidin, streptavidin, or neutra-avidin can be employed as the avidin protein. The conjugate can generally be prepared in a suitable buffer solution; this preparation proceeds rapidly at a temperature between room temperature and a temperature achieved with ice cooling.

Microparticles the surfaces of which have been modified with thyroxine or thyronine residues can be manufactured using the above conjugate. To manufacture the microparticles, it is generally preferable to employ microparticles the surfaces of which have been hydrophilically treated, such as polystyrene beads the surfaces of which have been hydrophilically treated with carboxyl groups. The diameter of the polystyrene beads is not specifically limited. For example, the diameter can be about 20 to 1,000 nm. Nor is the degree of hydrophilic treatment of the particle surfaces with carboxyl groups specifically limited. For example, the carboxyl groups are preferably present at a density of about 1 to 1,000 micromols/g. A hydration layer is formed on the surfaces of the particles when such microparticles are suspended in a suitable aqueous solvent, such as water, or a suitable buffer solution. The thickness of the hydration layer is not specifically limited. For example, the thickness can be about 5 to 50 nm. The presence and thickness of the hydration layer can be confirmed by a method such as dynamic light scattering (DSL). The type of microparticle is not specifically limited other than that a hydration layer forms on the surfaces of the microparticles when they are prepared as an aqueous dispersion. For example, in addition to polystyrene beads, a gold colloid, magnetic particles, fluorescent particles, Q dots (quantum dots), and the like can be employed.

When the conjugate obtained by reacting the compound represented by general formula (I) above with an avidin protein is reacted with the above-described microparticles, the avidin protein moiety of the conjugate is incorporated into the hydration layer with the hydrophobic thyroxine or thyronine residues protruding from the hydration layer. This immobilizes the thyroxine or thyronine residues on the surface of the microparticles. At the same time, aggregation of the microparticles can be inhibited as the hydration layer is present. A stable aqueous dispersion can thus be manufactured. For example, when employing polystyrene beads about 20 to 1,000 nm in particle diameter, the surfaces of which have been hydrophilically treated with carboxyl groups to a density of about 1 to 1,000 micromols/g, to form a hydration layer of about 50 to 500 nm, the number of avidin protein molecules in the above conjugate incorporated into the hydration layer will be about 100 to 6,400. A number of thyroxine or thyronine residues corresponding to this number is immobilized on the surface of the polystyrene beads. The concentration of the polystyrene beads in the aqueous suspension is not specifically limited. However, the concentration is about 0.0001 to 0.1 percent solid (“1 percent solid” meaning 1 g/10 mL), for example. The microparticles on the hydrophilically treated surfaces of which are immobilized avidin, streptavidin, or neutral avidin can be purchased as aqueous dispersions from Molecular Probes, Bangs Laboratories, or the like. The compound of general formula (I) above can be reacted with the aqueous dispersion to obtain an aqueous dispersion of microparticles with thyroxine or thyronine residues immobilized on the surface thereof. In that case, the aqueous dispersion is preferably diluted as needed with water or a suitable buffer solution such as PBS to prepare an aqueous dispersion with a microparticle solid content of 20 percent or lower.

The above aqueous dispersion can be employed to measure thyroxine or thyronine. The microparticles obtained as set forth above are maintained in a water-dispersible state by hydration of the protein, preventing aggregation, and can thus be suitably employed in thyroxine and thyronine bioassays. As a specific example of a bioassay, the method described in the Product Information provided by Molecular Probes, the disclosure of which is expressly incorporated by reference herein in its entirety, can be employed. However, the bioassay is not limited to the above examples. The aqueous dispersion of the present invention can be employed in immunochromatoassays, in which the aqueous dispersion is caused to seep into a film of nitrocellulose or the like; and ELISA assays in which the conjugate in the dispersion is immobilized on a solid-phase matrix such as polystyrene. However, potential applications are not limited to these methods.

In methods of measuring thyroxine or thyronine, optimal conditions are preferably determined in advance with the degree of surface modification of the microparticles with thyroxine or thyronine residues in the above-described aqueous dispersion as a variable.

The above-described conjugate in a form other than the above-described form of an aqueous dispersion can be used to measure thyroxine and thyronine and in analysis employing thyroxine and thyronine. In such measurement and analysis, for example, a method employing a material having a hydrophilically treated surface can be employed: the material may be modified with the above-described conjugate in the same manner as when employing the above-described microparticles. Examples of such methods are given below:

Kinetic analysis of antibodies against an immobilized antigen (T4 or T3) can be conducted by surface plasmon resonance (SPR). In SPR, for example, a sensor chip (CM5) can be employed in the form of a metal film the surface of which is covered with carboxymethyl dextran.

The purification of antibodies specific to T4 or T3 can be conducted with an affinity column using a support on which avidin has been immobilized.

Various analysis is possible by immobilizing T4 or T3 on chips on which avidin has been immobilized.

In the course of modifying a material having a surface that has been hydrophilically treated with the above-described conjugate, the conjugate that has been produced can be used to directly modify the material, or an avidin protein can be first immobilized on the material, after which the compound of the present invention represented by general formula (I) above can be added to form the conjugate on the material.

In methods of measuring thyroxine and thyronine, optimal conditions are preferably determined in advance with the degree of modification of the material with the conjugate as a variable.

### EXAMPLES

The present invention is described in greater detail below through embodiments. However, the scope of the present invention is not limited to the embodiments set forth below.

A spacer starting material in the form of polyethylene glycol chains was purchased from Quanta and alkyl chains were purchased from Watanabe Chemical Industries. Thyroxine and thyronine were purchased from Sigma-Aldrich. Biotin derivatives were purchased from Quanta and Sigma-Aldrich. p-Toluene sulfonic acid methyl ester (TsOMe), ethylenediamine, acetic anhydride (AC₂O), and Boc₂O were purchased from Wako Pure Chemical Industries and Tokyo Chemical Industry Co.

### Example 1

### Synthesis of Biotinylated Thyroxine (PEG n=4)

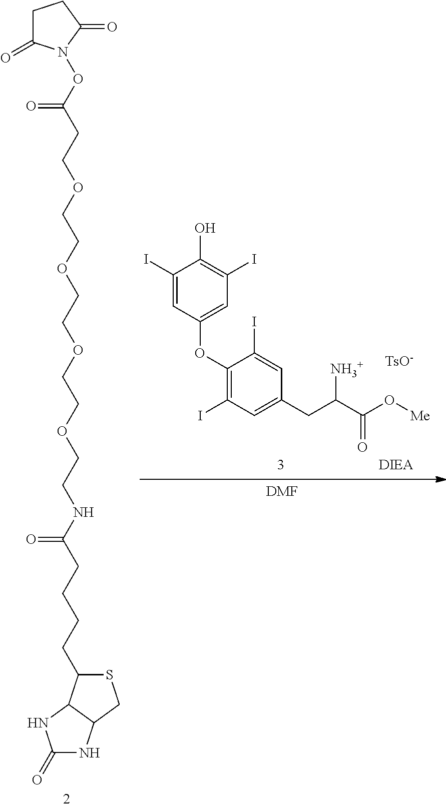

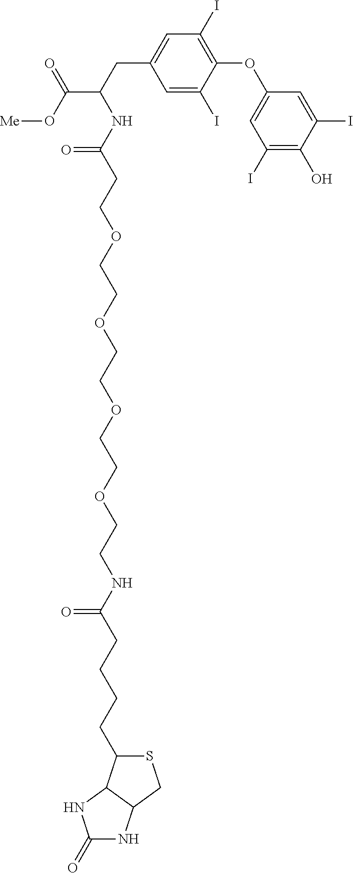

Compound 2 (300 mg, 0.51 mmol) and Compound 3 (590 mg, 0.61 mmol) were dissolved in 5 mL of DMF, 155 microliters (0.92 mmol) of DIEPA was added, and the mixture was stirred for 3 hours at room temperature. The completion of the reaction was confirmed by TLC. A 5 percent citric acid aqueous solution was added to the reaction mixture. The mixture was extracted three times with 20 mL of ethyl acetate and then dried with sodium sulfate. The solvent was distilled off under reduced pressure. The residue was purified by column chromatography (silica gel: CHCl₃ to CHCl₃/MeOH=20/1 to 10/1 to 6/1), yielding 200 mg (31 percent) of the target compound in the form of a white solid.

¹H-NMR (CD₃OD) δ/ppm; 7.81 (s, 2H), 7.06 (s, 2H), 4.70 (m, 1H), 4.45 (m, 1H), 4.25 (m, 1H), 3.78 (s, 3H), 3.60 (m, 10H) 3.20 (m, 2H), 2.90 (m, 2H), 2.70 (m, 1H), 2.20 (t, 2H), 1.20 to 1.60 (m, 6H)

ESI-MS (Positive): [M+1]⁺=1265

### Example 2

### (1) Preparation of Streptavidin-Modified Fluorescent Particles (210 nm in Diameter)

To 250 microliters of a 2 percent fluorescent particle aqueous dispersion (F8811, 210 nm in diameter, Molecular Probes: Yellow-green (505/515)) were added 150 microliters of 50 mM MES buffer (pH 6.0) and 100 microliters of 10.0 mg/mL streptavidin PBS solution, and the mixture was stirred for 15 minutes at room temperature. A 5 microliter quantity of 400 mg/mL WSC (product number 01-62-0011, Wako Pure Chemical Industries) aqueous solution was added and the mixture was stirred for 2 hours at room temperature. A 25 microliter quantity of 2 mol/L glycine aqueous solution was added, the mixture was stirred for 30 minutes, and the mixture was centrifugally separated (15,000 rpm, 4° C., 15 minutes) to cause the particles to precipitate out. The supernatant was removed, 500 microliters of PBS (pH 7.4) was added, and an ultrasonic cleaning device was used to redisperse the fluorescent particles. Centrifugal separation was conducted again (15,000 rpm, 4° C., 15 minutes), the supernatant was removed, 500 microliters of a 1 percent BSA PBS (pH 7.4) solution was added, and the fluorescent particles were redispersed, yielding a 1 percent (w/v) aqueous dispersion of streptavidin-modified fluorescent particles.

(2) Preparation of Bound Biotinylated Thyroxine (PEG n=4) Fluorescent Particles

The biotinylated thyroxine (PEG n=4) obtained in Example 1 and the streptavidin-modified fluorescent particles prepared in (1) above were mixed to achieve the concentration ratio indicated in Table 1 and the mixture was reacted for 15 hours at room temperature. The particles were precipitated by centrifugal separation (15,000 rpm, 4° C., 15 minutes), the supernatant was removed, 500 microliters of PBS (pH 7.4) was added, and the fluorescent particles were redispersed with an ultrasonic cleaning device. Centrifugal separation (15,000 rpm, 4° C., 15 minutes) was further conducted, the supernatant was removed, 500 microliters of a 1 percent BSA PBS (pH 7.4) solution was added, and the fluorescent particles were redispersed to obtain an aqueous dispersion of biotinylated thyroxine (PEG n=4)-bound fluorescent particles.

TABLE 1

| | Streptavidin-modified | Biotinylated | Blending ratio |
|---|---|---|---|
| | fluorescent particles | thyroxine (PEG | (molecules/ |
| | (weight %) | n = 4) (mol/L) | particle) |
| Conditions A | 0.5 | 7.9E−05 | 48480 |
| Conditions B | | 2.5E−05 | 15150 |
| Conditions C | | 1.2E−05 | 7575 |
| Conditions D | | 6.2E−06 | 3787 |
| Conditions E | | 3.1E−06 | 1894 |
| Conditions F | | 1.5E−06 | 947 |
| Conditions G | | 7.7E−07 | 473 |
| Conditions H | | 3.9E−07 | 237 |
| Conditions I | | 1.9E−07 | 118 |
| Conditions J | | 9.7E−08 | 59 |

(3) Preparation of an Anti-Thyroxine Antibody Solid-Phase Plate

A 100 microliter quantity of 150 mM sodium hydrochloride solution of anti-thyroxine monoclonal antibody (#100074, Medix) adjusted to 10 micrograms/mL was added to each well of a 96-well black plate (#475515, NUNC) and the solution was left standing for 1 hour at room temperature. The antibody solution was removed and cleaning was conducted (350 microliters/well, 3 times) with a cleaning buffer (PBS (pH 7.4) containing 0.05 percent (w/v) Tween-20) prepared in advance. When the cleaning was completed, 200 microliters of PBS (pH 7.4) containing 1 percent casein was added to each well to block the portion not adsorbed with antibody, and the solution was left standing at room temperature for 3 hours. Following cleaning with the above cleaning buffer, 200 microliters of a stabilizing agent in the form of Immunoassay Stabilizer (ABI) was added to each well, the solution was left standing for 30 minutes at room temperature, the solvent was removed, and the moisture was completely removed in a drying device. The product obtained was employed for testing.

(4) Competitive Immunoassay

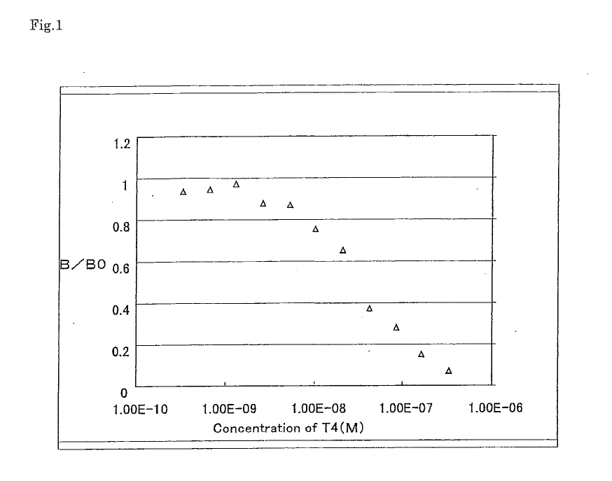

The bound biotinylated thyroxine (PEG n=4) fluorescent particles prepared under conditions D were diluted to 0.005 weight percent with PBS solution (pH 7.4) containing 1 weight percent BSA. A dilution series was prepared in the concentrations indicated below from 26 microgram/dL thyroxine solution with phosphate buffer solution (pH 7.4) (FIG. 1), and employed as antigen solution. A 30 microliter quantity of 0.005 weight percent biotinylated thyroxine (PEG n=4)-bound fluorescent particles was mixed with 30 microliters of each of the various levels of thyroxine solutions. Out of each solution, 50 microliters each was added to the individual wells of an anti-thyroxine antibody solid-phase plate. After allowing the wells to stand at room temperature for one hour, the reaction solutions were removed. Cleaning was conducted with 350 microliters of phosphate buffer solution (pH 7.4), after which the fluorescent intensity was measured with a microplate reader (ARVOMX, PerkinElmer). The measurement results are given in FIG. 1. The results indicated that highly sensitive measurement of T4 was possible with the compound of the present invention.

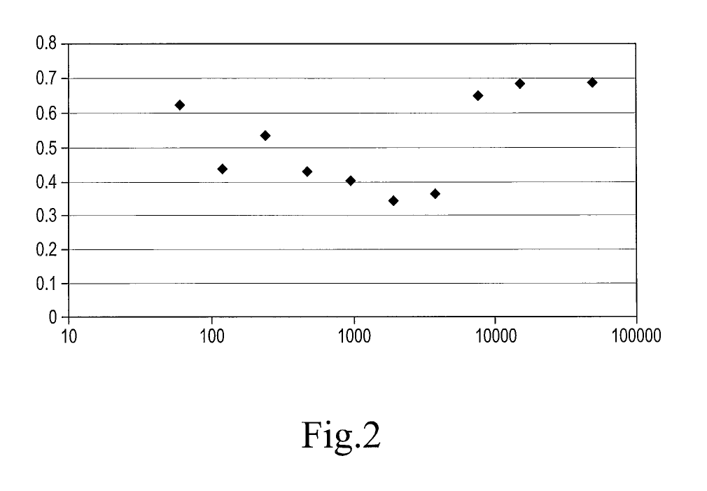

Further, biotinylated thyroxine (PEG n=4)-bound fluorescent particles prepared under conditions A to J were similarly evaluated to determine whether or not the optimal labeling rate could be controlled in a competitive assay simply by changing the blending ratio of biotinylated T4 and the particles. FIG. 2 is a plot of the blending ratio and the reaction blocking rate at a T4 concentration of 1.5 e⁻⁷M. In the figure, the blending ratio (labeling rate=amount of antigen/particles) is plotted on the horizontal axis and the reaction blocking rate on the vertical axis. In a competitive immunoassay, it is necessary to set conditions at which the blocking rate of the labeled antigen and the antigen are maximized. When the fluorescent particles were labeled with the compound (PEG n=4) obtained in Example 1, the particle concentration was kept constant, and the amount of antigen was varied, it will be understood that the blocking rate peaked in the vicinity of a labeling rate of 40,000. On the other hand, when examining assay conditions in this manner by a method of immobilizing antigen such as the carboimide method (chemical bonding), it is difficult to control the number, thus complicating the search for conditions at which the blocking rate becomes maximum. Accordingly, these results indicate that the compound of the present invention is extremely useful for measuring thyroxine.

### Example 3

### (1) Preparing Biotinylated Thyroxine—Streptavidin Conjugate

The biotinylated thyroxine obtained in Example 1 (PEG n=4) (Biotin-PEG₄-T4) (1.2 mg) was dissolved in 189.8 microliters of DMSO.

A 98 microliter quantity of 1 mg/mL of streptavidin and 1.33 microliters of the DMSO solution (5 mM) of Biotin-PEG₄-T4 obtained above were dissolved in 0.67 microliters of HBSN. Here, the streptavidin:biotin ratio was 1:4 (mole ratio).

(2) Preparation of an SPR Flow Cell

A Biocore 3000 (Biocore) was employed as the surface plasmon resonance (SPR) device. CM5 sensor chips were employed, and four flow cells (Fc 1 to 4) were prepared according to the following procedure. The flow rate was 10 microliters/min.

Fc1: Streptavidin (st-Avidine) Immobilized Flow Cell

A flow of a 70 microliter quantity of a mixed solution of equal quantities of EDC (1-ethyl-3-(3-dimethylaminopropyl)carbodiimide) and NHS (N-hydroxysuccinimide) was run (7 minutes), after which a flow of a 70 microliter quantity of a solution of streptavidin in dimethylsulfoxide (DMSO) (0.2 mg/mL, pH 5.0) was run (7 minutes). Subsequently, a flow of ethanolamine was similarly run.

### Fc2, 3, 4: T4-Streptavidin Immobilized Flow Cells

A flow of a 70 microliter quantity of mixed solution of equal quantities of EDC and NHS was run (7 minutes), after which a flow of a 70 microliter solution of T4-streptavidin in DMSO (0.2 mg/mL, pH 5.0) was run (7 minutes). Subsequently, a flow of ethanolamine was similarly run.

When the levels of immobilization were checked, measurement levels of 1,000 RU, 1,000 RU, 400 RU, and 100 RU were obtained for Fc1, Fc2, Fc3, and Fc4, respectively.

It was necessary to conduct interaction analysis at the minimum level of immobilization within the range over which the S/N ratio could be calculated. Thus, Fc3, with a level of immobilization of 400 RU, was employed in analysis as the T4-streptavidin immobilization flow cell. Fc1 was employed as a negative control.

### (3) Preparation of Antibody Solution

Antibodies made by Medix (Antibody 1), two types B and C (Antibodies 2 and 3) made by Fitzgeraldx, and East Coast Bio (Antibody 4) were employed as the antibodies.

Solution concentrations of 0.2, 1, 5, and 25 nM of each antibody were prepared and measurement was conducted. The 1, 5, and 25 nm concentrations were employed in data analysis.

(4) Evaluation by SPR

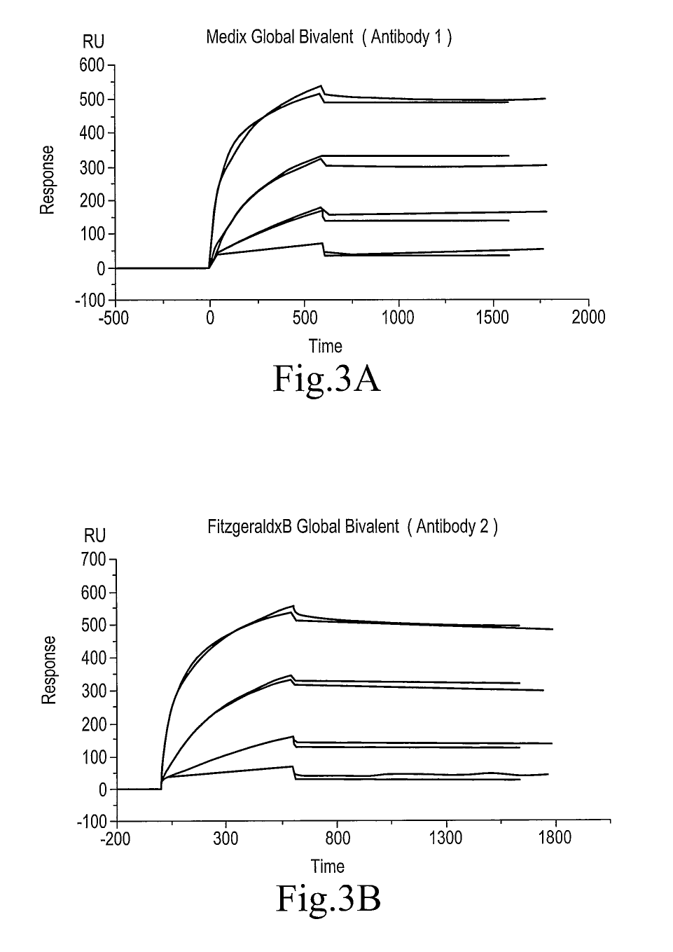

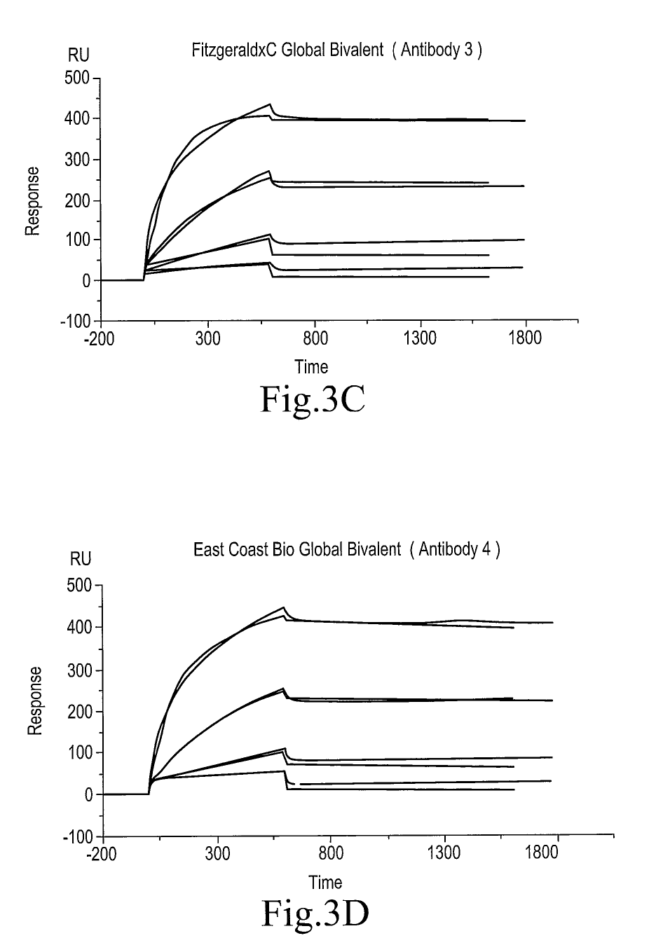

Evaluation was conducted with Fc1 and Fc3 under the following conditions by adding the antibody solutions. The measurement results are given in FIG. 3. The results of bivalent (bivalent bond interaction) kinetic analysis are given in Table 2.

### Flow Rate, Binding, Dissociation, and Cleaning Conditions

Flow: 10 microliters/mL

### Binding: 10 min

### Dissociation: 20 min

### Cleaning 1 Gly pH 1.5: 3 min

### Cleaning 2 Gly 10 mM NaOH: 3 min

### Cleaning 3 Gly pH 1.5: 3 min

### Cleaning 4 Gly 10 mM NaOH: 3 min

Regeneration: HBSP buffer 8 min

TABLE 2

| | | ka | kd | | Sub- |
|---|---|---|---|---|---|
| Sample | K_{d} | M⁻¹ s⁻¹ | s⁻¹ | Clone | class |
| Antibody 1 | <3.7 × 10⁻¹¹ | 2.7 × 10⁵ | <10⁻⁵ | — | IgG₁ |
| Antibody 2 | 1.6 × 10⁻¹⁰ | 2.2 × 10⁵ | 3.5 × 10⁻⁵ | M94207C | IgG₁ |
| Antibody 3 | 1.5 × 10⁻⁸ | 7.9 × 10⁴ | 1.2 × 10⁻³ | M94208 | IgG_{2beta} |
| Antibody 4 | 5.2 × 10⁻¹⁰ | 7.9 × 10⁴ | 4.1 × 10⁻⁵ | — | IgG_{2b} |

From the results in Table 2, it will be understood that antibody 1 had the greatest compatibility. Antibodies 1 and 2 exhibited about the same binding rates.

### Example 4

### Synthesis of Biotinylated Thyronine (PEG n=4)

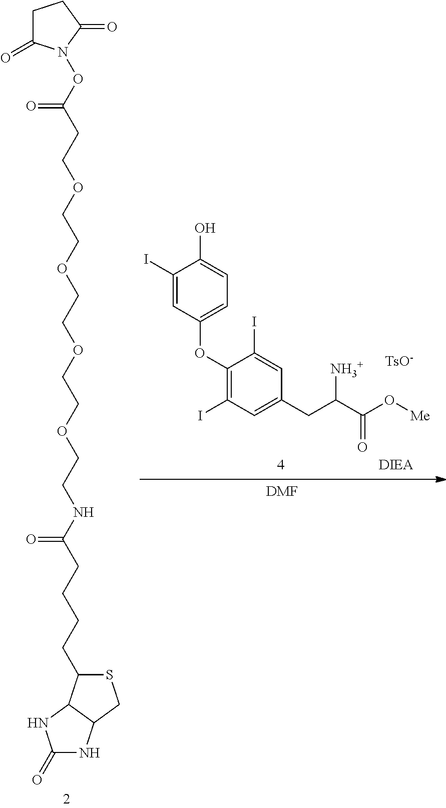

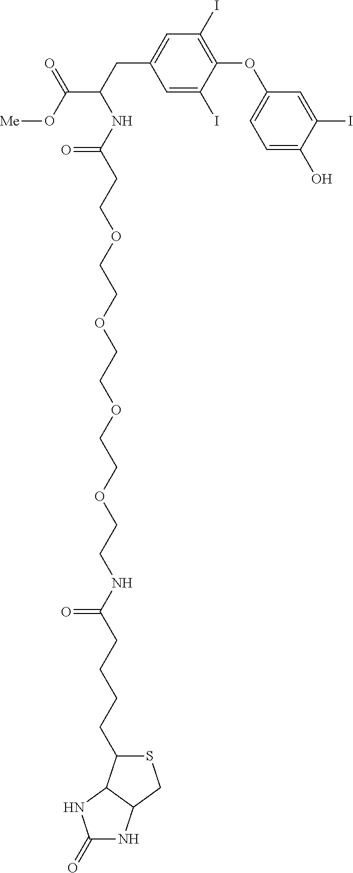

Compound 2 (300 mg, 0.51 mmol) and Compound 4 (590 mg, 0.61 mmol) were dissolved in 5 mL of DMF, 155 microliters (0.92 mmol) of DIEPA was added, and the mixture was stirred for 3 hours at room temperature. The completion of the reaction was confirmed by TLC. A 5 percent citric acid aqueous solution was added to the system and the mixture was extracted 3 times with 20 mL of ethyl acetate. Subsequently, the mixture was dried with sodium sulfate, the solvent was distilled off under reduced pressure, and the residue was purified by column chromatography (silica gel: CHCl₃ to CHCl₈/MeOH=20/1 to 10/1 to 5/1), yielding 200 mg (31 percent) of a white solid. Identification was subsequently conducted by ¹H-NMR. The results are given below.

¹H-NMR (CD₃OD) δ/ppm; 7.81 (s, 2H), 6.99 (d, 1H), 6.75 (d, 1H), 6.60 (dd, 1H) 4.75 (m, 1H), 4.50 (m, 1H), 4.25 (m, 1H), 3.78 (s, 3H), 3.60 (m, 10H) 3.20 (m, 2H), 2.90 (m, 2H), 2.70 (d, 1H), 2.20 (t, 2H), 1.40 to 1.80 (m, 6H)

ESI-MS (Positive): [M+1]⁺=1139.4

### INDUSTRIAL APPLICABILITY

The compound of the present invention is useful in the measurement of thyroxine and thyronine. For example, a conjugate obtained by binding the compound of the present invention to an avidin protein can be used to modify the surface of microparticles such as polystyrene beads that have been hydrophilically surface treated with carboxyl groups, for example. Since microparticles obtained in this fashion do not aggregate due to the effect of protein hydration, the microparticles are suitable for use in thyroxine and thyronine bioassays, permitting the convenient and accurate measurement of thyroxine and thyronine with good reproducibility.

Further, the compound of the present invention can be used as a means of providing immobilized thyroxine or thyronine, and thus used in the measurement and analysis of thyroxine and thyronine antibodies.

What is claimed is:

1. A compound represented by general formula (I) below: 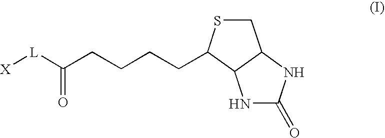
wherein L represents a linear or branched-chain linker having 5 to 50 total atoms; X represents one or more thyroxines, thyronines, or residues of derivatives of thyroxine or thyronine, bound to the main chain and/or side chain of the linker represented by L.

2. The compound according to claim 1, in which the linker represented by L comprises at least one chain-like structure selected from the group consisting of an alkyl chain, a polyethylene glycol chain, and a peptide chain.

3. The compound according to claim 2, in which L comprises one or more bonds selected from the group consisting of an amide bond, an ester bond, and a carbamate bond in addition to an alkyl chain and/or a polyethylene glycol chain.

4. The compound according to claim 1, in which the main chain of the linker represented by L consists of 15 to 20 atoms.

5. The compound according to claim 1, in which the main chain of the linker represented by L is 25 to 30 Angstroms in total length.

6. The compound according to claim 1, wherein the residue is a residue obtained by removing the hydrogen atom from a carboxyl group of thyroxine, thyronine, or a derivative of thyroxine or thyronine, or a residue obtained by removing one of the hydrogen atoms in the amino group of thyroxine or thyronine.

7. The compound according to claim 1, wherein the derivative is a carboxylic acid ester derivative or an amino group-modified derivative.

8. The compound according to claim 1, having an interaction with an avidin protein represented by a Kd value of 1×10⁻¹³ or lower.

9. A compound represented by the following formula: 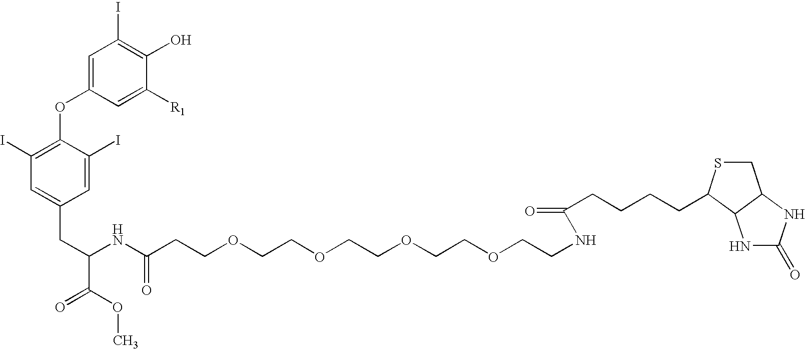
wherein R₁ represents hydrogen atom or iodine atom.

10. A conjugate in the form of the compound represented by general formula (I) set forth in claim 1 bonded to an avidin protein.

11. The conjugate according to claim 10, wherein the avidin protein is avidin, streptavidin, or neutra-avidin.

12. An aqueous dispersion comprising a microparticle the surface of which is hydrophilically treated, the surface of said microparticle being modified with the conjugate set forth in claim 10.

13. The aqueous dispersion according to claim 12, wherein the microparticle is a carboxy hydrophilically-treated polystyrene bead.

14. The aqueous dispersion according to claim 13, wherein the avidin protein moiety of the conjugate is contained in a hydration layer formed on the surface of the carboxy hydrophilically-treated polystyrene bead.

15. The aqueous dispersion according to claim 12 employed for a measurement of thyroxine or thyronine.

16. A method for measuring thyroxine or thyronine using the aqueous dispersion set forth in claim 12.
